(19) United States
(12) Patent Application Publication
(10) Pub. No.: US 2021/0106290 A1
(43) Pub. Date: Apr. 15, 2021

(54) SYSTEMS AND METHODS FOR THE DETERMINATION OF AROUSAL STATES, CALIBRATED COMMUNICATION SIGNALS AND MONITORING AROUSAL STATES

(71) Applicant: TECHNOLOGIES HOP-CHILD, INC., Sherbrooke (CA)

(72) Inventors: Diane GROLEAU, Ste-Catherine de Hatley (CA); Pascale BROCHU, Sherbrooke (CA); Marc-Antoine PELLETIER, Georgeville (CA); Simon CORCOS, Verdun (CA); Alex SALVAIL, Sherbrooke (CA); Julien DUCHESNE, Sherbooke (CA); Rozarina Md Yusof HOWTON, Calgary (CA)

(21) Appl. No.: 17/072,145

(22) Filed: Oct. 16, 2020

Related U.S. Application Data
(63) Continuation of application No. PCT/CA2019/050473, filed on Apr. 16, 2019.
(60) Provisional application No. 62/658,353, filed on Apr. 16, 2018.

(51) Int. Cl. A61B 5/00; A61B 5/024; A61B 5/026; A61B 5/145; A61B 5/11; A61B 5/24; A61B 5/01; A61B 5/08; G16H 50/30

(52) U.S. Cl. CPC A61B 5/7475; A61B 2560/0223; A61B 5/024; A61B 5/026; A61B 5/14517; A61B 5/11; A61B 5/24; A61B 5/01; A61B 5/082; A61B 5/4088; A61B 5/7455; A61B 5/7405; G16H 50/30; A61B 5/742; A61B 5/7264; A61B 5/6801

(57) ABSTRACT

A computer-implemented method and system for determining an arousal state of a user, the method comprising: obtaining input data relating to a response of the user to a communication signal, wherein the input data relates to one or more of: a direct response of the user, a physiological response of the user, the physiological response comprising at least one measured physiological value, and determining the arousal state of the user based on the input data, and one or more of: contextual data, an initial arousal state of the user, and user profiles. A computer-implemented method and system for determining a calibration communication signal for arousal state modulation. A computer-implemented method and system for monitoring a user's arousal state.

### FIELD

The present technology relates to systems and methods for the determination of arousal states of a user, calibrated communication signals to apply to the user, and monitoring of arousal states of the user, as well as uses of the determined arousal states, calibrated communication signals, and monitored arousal states.

### BACKGROUND

Communication signals as sensory inputs include any type of signal that can be perceived by a user's sense and for the purposes of conveying information or for interacting in any way. Examples of such communication signals include, but are not limited to, one or more of speech (sense of hearing), still or moving images (sense of sight), and haptic (sense of touch). Communication signals can also be referred to as interaction signals.

Communication signals can be defined by a number of different parameters. For example, in speech as a communication signal, the parameters can include any one or more of the lexicon, the tone, the pitch, the loudness, the speed and the duration of the speech. Varying any one or more of these parameters can convey different information.

However, each user differs from one another in their processing ability of sensory inputs. Therefore, the same communication signal can convey different information to different users.

This variability in sensory input processing ability is often more pronounced in certain portions of the population, such as those users with cognitive or neurological disorders such as autism. For such users, the same communication signal can communicate different things on different occasions, and a one-size fits all approach is wholly ineffective.

The perception of a given communication signal by a particular user may be further confounded by the arousal state of that user at any particular moment in time.

Therefore, there is a need for systems and methods for determining arousal states of a user and communication signals that are tailored to the user.

### SUMMARY

Embodiments of the present technology have been developed based on developers' appreciation of shortcomings associated with the prior art.

In particular, such shortcomings may comprise (1) limited ability to convey a desired communication to the same or different users by using the same communication signal; (2) limited ability to determine an appropriately calibrated communication signal for the user; and/or (3) limited ability to determine when to provide a calibrated communication signal to the user.

Developers have identified that, in certain embodiments, the ability to determine an arousal state (also referred to as “cognitive arousal state”, “current arousal state” or “user arousal state”) of the user may be helpful for: (1) determining an appropriately calibrated communication signal for the user, (2) monitoring the user to determine when to provide a calibrated communication signal, and (3) evaluating effects of certain treatments and therapies on the user. By “therapy” is meant treatment or caregiving, whether physical, chemical or mental in nature.

By “arousal state” is generally meant a state of sensory alertness, mobility, and readiness to respond. Without being held to any theory, the arousal state involves the ascending reticular activating system (ARAS) in the brain, which mediates wakefulness, the autonomic nervous system, and the endocrine system. Arousal states may be classified as HIGH, MID and LOW, or in any other manner For example, arousal states may be defined in terms relative to adverse states such as emotional dysregulation (also referred to as “melt-downs” or “crises”). Arousal states may be distinct from emotions.

By “communication signal” is meant any signal that can be used as a sensory input, such as to convey information to a user, to provoke a reaction from the user, or to interact with the user in any way. In certain embodiments, communication signals comprise any type of signal that can affect the central nervous system of the user and the brain.

By “calibrated communication signal” is meant a communication signal which is tailored to a specific user, a specific group of users, or for specific purposes including, but not limited to, arousal state modulation or the communication of certain information. The calibrated communication signal may also be task specific to the user.

In certain embodiments, aspects of the present technology allow the more accurate determination of the arousal state of the user or allow a faster determination of the arousal state of the user. This in turn may allow for a more efficient determination of the calibrated communication signal, a more timely intervention (such as before or during the dysregulation event), a more efficient communication with the user, a more efficient monitoring of the user, and/or a more efficient treatment or therapy evaluation. In certain embodiments, developers have noted that the calibrated communication signal can be used for arousal state modulation (or “arousal state regulation”). By modulation/regulation is meant a change in the current arousal state to a different arousal state, a maintenance in the current arousal state, a change within the current arousal state, and a change in the amount of time spent in that arousal state.

Various aspects and embodiments of methods and systems of the present technology may be applied to users with a cognitive or neurological disorder such as autism spectrum disorder (ASD), or ADHD (Attention Deficit and Hyperactivity Disorder).

From a broad aspect, the present technology relates to methods and systems for determining arousal states based on user feedback to communication signals and determined according to user profiles.

From one aspect, there is provided a method for determining a current arousal state of a user, the method executed by a processor of a computer system, the method comprising: obtaining input data relating to a response of the user to a communication signal, wherein the input data relates to one or both of: a direct response of the user to the communication signal, and a physiological response of the user to the communication signal, and determining the current arousal state of the user based on: the input data of the user; one or more of: contextual data regarding current contextual factors relating to different available arousal states of the user, and an initial arousal state of the user; and a user profile of the user, wherein the user profile includes data defining a relationship between the input data, one or more of the contextual data and the initial arousal state, and different available arousal states of the user.

In certain embodiments, the determining of the current arousal state comprises determining, by a trained machine learning algorithm the current arousal state of the user, the machine learning algorithm trained to determine the current arousal state of the user based on at least one of: the input data, the user profile data, an initial arousal state of the user, and the contextual data, wherein the user profile data comprises data relating to one or more of: a physiological parameter profile of the user, a disorder profile of the user, a sensory profile of the user, a contextual factor profile of the user, an interaction profile of the user, optionally the user profile data including contextual factor weights for the user, and physiological parameter weights for the user.

The physiological parameter profile may define physiological value ranges of one or more physiological parameters when the user is within a certain arousal state. The at least one physiological parameter may have discrete or overlapping value ranges in one or more arousal states. The physiological parameter weights may comprise relative weights of the physiological parameters as an indication of its pertinence to an arousal state. An initial arousal state of the user may be a preliminary arousal state which requires correcting, validating, or fine-tuning. The initial arousal state may be defined by the user, by an other user, or by the processor based on initial parameters. The disorder profile comprises information regarding a disorder level of the user, if applicable. The sensory profile of the user comprises information on sensory processing patterns of the user. The contextual factor profile comprises information relating to the effect of one or more contextual factors on the sensory profile of the user. The contextual factor profile may also comprise information relating to the effect of one or more contextual factors on the disorder profile of the user. Contextual factors may comprise external influences such as the environment, tiredness, medication, etc. The interaction profile may define value ranges of interaction parameters with at least one arousal state of the user. Contextual factor weights may comprise relative weights of the contextual factors as an indication of its pertinence to the sensory profile of the user. Contextual data may comprise contextual factor values. In certain embodiments, the user profile (e.g. one or more of the physiological parameter profile, the physiological parameter weights, the initial arousal state, the disorder profile, the sensory profile, the contextual factor weights, the interaction profile) are user specific, and may be at least partially predetermined. The user profile may be stored in one or more databases, which may be updated dynamically.

In certain embodiments, the obtaining the input data relating to the response of the user to the communication signal comprises receiving the input data relating to the response of the user from at least one of: a communication device, the communication device being a wearable device associated with the user and operably connected to the processor, an input device associated with the user or with an other user, and the other user. The direct response of the user may comprise one or more of a touch response, a sound response, a kinetic response, a brain signal response, a breath response, and a facial response from the user in reaction to the communication signal. The physiological response of the user to the communication signal may comprise one or more of a heart rate, a breathing rate, a blood flow, a sweat analysis, a measure of movement, an electrical brain signal, a temperature, a breath analysis, and one or more biomarkers of stress.

In certain embodiments, the method is implemented by a processor in communication with one or more input devices associated with the user. The one or more input devices can be communicatively connected to the processor. In certain embodiments, input data is obtained from an input device associated with the user, such as a sensor for obtaining physiological data or an interface for capturing the direct response of the user. In certain embodiments, the input data is obtained from the other user observing the user's reaction to the communication signal, such as through an input device (e.g. a monitoring device) associated with the other user. In certain embodiments, the input device associated with the user may be one or more wearable devices arranged to measure one or more of movement, blood flow, and sweat of the user, and having a touch screen for receiving the direct user response.

In certain embodiments, the obtaining the input data relating to the direct response of the user to the communication signal comprises: obtaining user input values of the direct response of the user, the user input values relating to one or more user input parameters, the user input parameters including one or more of: an intensity of the direct user response, a duration of the direct user response, a time delay of the direct user response, a location of the direct user response, a frequency of the direct user response, and a pattern of when the responses are given over a known time period.

In certain embodiments, the determining the current arousal state comprises comparing/assessing the direct response of the user to a weight allocation of the given user input parameter, the user input parameter comprising at least one of: an intensity of the direct user response, a duration of the direct user response, a time delay of the direct user response, frequency of the direct user response, and variations in the pattern of when the responses are given over a known period of time.

In certain embodiments, the current arousal state comprises one of a plurality of available arousal states categories, the method comprising determining a user index, the user index being indicative of a given arousal state category and a relative position within the arousal state category.

In certain embodiments, the method further comprises sending instructions to a communication device to provide the communication signal to the user, the communication device being a wearable device associated with the user and operably connected to the processor, wherein the communication signal is a haptic signal, the haptic signal being defined by one or more of: a signal amplitude, a signal frequency, a signal wavelength, a signal duration, a signal pattern, and a signal code.

In certain embodiments, the method further comprises sending instructions to two communication devices, the two communication devices comprising two haptic devices arranged to provide a bilateral signal to the user.

In certain embodiments, the method further comprises determining the communication signal to provide to the user, the determining the communication signal being based on the user profile, and one or more of: the initial arousal state of the user, the contextual data, and a preference of the user.

In certain embodiments, the communication signal is a frequency-based signal defined by one or more of: a signal type, a signal amplitude, a signal frequency, a signal wavelength, a signal duration, a signal code and a signal pattern. The determining the communication signal to provide to the user may comprise determining one or more of: the signal type, the signal amplitude, the signal frequency, the signal wavelength, the signal duration, the signal code and the signal pattern.

In certain embodiments, the method further comprises obtaining input of a desired arousal state for the user; and determining whether a modulation in the user's current arousal state is required by comparing the determined current arousal state with the desired arousal state.

In certain embodiments, the method further comprises determining a calibrated communication signal effective to modulate the user from the determined arousal state to the desired arousal state, the determining the calibrated communication being based on the user profile of the user including the baseline data. The user profile may include one or more of a sensory profile of the user, a contextual factor profile of the user, a contextual factor weights for the user, a physiological parameter profile, physiological parameter weight, a disorder profile of the user, an interaction profile of the user, and a response of the user to a validation communication signal.

In certain embodiments, the determining the calibrated communication signal comprises executing, by the processor, a trained machine learning algorithm, the machine learning algorithm having been trained on at least one of the following training inputs: the determined arousal state of the user, the desired arousal state of the user, the disorder profile of the user, the interaction data profile, the sensory profile, the contextual factor profile, the contextual factor weights, the physiological parameter profile, the physiological parameter weights, and the input data.

In certain embodiments, the calibrated communication signal is one or more of: (i) a haptic signal; (ii) a light signal; (iii) a sound signal; (iv) an olfactory signal; (v) a visual kinetic signal, (vi) a sensory kinetic signal, (vii) a magnetic signal, (viii) an electric brain signal, and (ix) a piezometric signal.

In certain embodiments, the method further comprises sending instructions to a communication device associated with the user, or to an input device associated with the user, to apply the calibrated communication signal. The calibrated communication signal may comprise an action, a spoken word, or other communication signal, optionally delivered by a virtual character depicted on a screen of the input device or the communication device.

In certain embodiments, the determining the calibrated communication signal comprises the processor receiving a desired communication to be communicated to the user from another user of the system, transforming the desired communication using parameters determined by the calibrated communication signal, and sending instructions to the input device or the communication device to provide transformed desired communication. The desired communication may be specific to a task to be undertaken by the user.

In certain embodiments, the method further comprises obtaining input data relating to the user in response to the calibrated communication signal, and adjusting at least one parameter associated with the calibrated communication signal until the desired arousal state is achieved.

In certain embodiments, the method further comprises applying the calibrated communication signal until one or more of: the direct response represents a desire to stop; the physiological response reaches a predetermined threshold (for example, the heart rate or the blood flow reaches a predetermined threshold value); a predetermined time has lapsed; a fail-safe level is reached or exceeded, and the user self-regulates. In certain embodiments, the physiological response can be indicators of undue stress, overload or discomfort of the user. In certain embodiments, the predetermined threshold can be measured, such as by the input device, or observed by another person / user 2. In certain embodiments, the physiological response is as determined by one or more of physiological stress markers (e.g. cortisol level); unresponsiveness of the user for a predetermined time period; and extreme body temperature of the user or a sensation of extreme body temperature by the user (e.g. cold).

In certain embodiments, the method comprises sending instructions to the communication device to end the application of the calibrated communication signal when the predetermined threshold end value is reached. If this is not reached, the method continues applying the calibrated communication signal until the direct response representing the desire to stop is received. If the direct response is not received, the method continues applying the calibrated communication signal until the fail-safe level is reached. In any of the end scenarios, particularly if the fail-safe level end, the method comprises the processor sending instructions to the communication device to end the calibrated communication signal as quickly as possible. In certain other embodiments, these method steps can be performed in any order, as long as the fail-safe level step is included.

In certain embodiments, the method comprises storing the determined arousal state in a database or providing it as an output on an electronic device associated with the user or the other user.

In certain embodiments, the method further comprises monitoring physiological data associated with the user, and when a predetermined trigger is noted, determining the communication signal or the calibrated communication signal, and optionally sending instructions to the communication device to provide the communication signal or the calibrated communication signal to the user. The monitoring may be continuous and in real-time. In certain embodiments, the monitoring of the user is remote from the user, such as by a caregiver.

In certain embodiments, the method further comprises storing in one or more databases communicatively coupled to the processor, any one or more of the user profile, associated parameters of the user profile, associated data of the user profiles, and associated weights of the user profile parameters. The user profile may comprise, but is not limited to, a sensory profile of the user, a contextual factor profile of the user, a physiological parameter profile of the user, contextual factor weights, physiological parameter profile weights, the disorder profile, the interaction profile, the trained machine learning algorithm, predetermined threshold data, input data, and contextual data, the determined arousal state in response to the communication signal, the calibrated communication signal, validated communication signal and the baseline data.

In certain embodiments, the user has a cognitive or neurological disorder, such as but not limited to autistic spectrum disorder.

From another aspect, there is provided a system for determining an arousal state of a user, the system comprising: a computer system having a processor, the processor arranged to: obtain input data relating to a response of the user to a communication signal, wherein the input data relates to one or both of: a direct response of the user to the communication signal, the direct response comprising at least one measured direct response value, and a physiological response of the user to the communication signal, the physiological response comprising at least one measured physiological value, and determine the arousal state of the user based on: the input data of the user; one or more of: contextual data regarding current contextual factors relevant to the arousal state of the user, and an initial arousal state of the user; and a user profile of the user; wherein the user profile includes baseline data defining a relationship between the input data, one or more of the contextual data and the initial arousal state, and the arousal state of the user.

In certain embodiments, the system further comprises an input device, operably connected to the processor, for obtaining the input data relating to the response of the user to the communication signal, the input device comprising a wearable device arranged to measure one or more of: a direct response comprising one or more of a touch response, a sound response, a kinetic response, a brain signal response, a breath response, and a facial response from the user in reaction to the communication signal, and a physiological response comprising one or more of a heart rate, a breathing rate, a blood flow, a sweat analysis, a measure of movement, an electrical brain signal, a temperature, a breath analysis, and one or more biomarkers of stress. The system may comprise another input device, or the same input device, for measuring contextual data.

In certain embodiments, the system further comprises a communication device, operably connected to the processor and optionally to the input device, for providing the communication signal to the user. The input device and the communication device can be implemented as a wearable device associated with the user.

In certain embodiments, the system further comprises a monitoring device, operably connected to the processor, and optionally to one or both of the input device and the communication device, for providing a calibrated communication signal to the user, the calibrated communication signal being effective to modulate the user from the determined arousal state to a desired arousal state. The monitoring device may comprise an electronic device having a screen for displaying an avatar to provide the calibrated communication signal to the user.

From another aspect, there is provided a method for determining a calibrated communication signal for arousal state modulation of a user, the method executed by a processor of a computer system, the method comprising: obtaining input of a current arousal state of the user; obtaining input of a desired arousal state for the user; determining a required modulation to the arousal state of the user to achieve the desired arousal state or to maintain the current arousal state; and determining the calibrated communication signal effective to achieve the required modulation for the user or to maintain the current arousal state. The calibrated communication signal can also be determined for purposes other than arousal state modulation, such as for the communication of certain information to the user or for other interactions withe the user.

In certain embodiments, the determining the calibrated communication signal is based on one or more of a user profile, contextual data relating to environmental factors relevant to the arousal state of the user, a response of the user to a validation communication signal, and optionally wherein the user profile comprises at least one of a sensory profile of the user, a disorder profile of the user, a contextual factor profile of the user, contextual factor weights, an interaction profile of the user, a physiological parameter profile, physiological parameter weights.

In certain embodiments, the determining the calibrated communication signal comprises executing, by the processor, a trained machine learning algorithm, the machine learning algorithm having been trained on one or more of the following training inputs: the arousal state of the user, a desired arousal state of the user, a disorder profile of the user, an interaction data profile of the user, a sensory profile of the user, a contextual factor profile of the user, contextual factor weights of the user, the physiological parameter profile of the user, the physiological parameter weights of the user, and a response of the user to a validation communication signal.

In certain embodiments, the obtaining the input of an arousal state of the user comprises executing the any steps or aspects of the abovementioned method.

In certain embodiments, the calibrated communication signal is one or more of: (i) a haptic signal; (ii) a light signal; (iii) a sound signal; (iv) an olfactory signal; (v) a visual kinetic signal, (vi) a sensory kinetic signal, (vii) a magnetic signal, (viii) an electric brain signal, and (ix) a piezometric signal.

In certain embodiments, the calibrated communication signal comprises a frequency-based signal defined by one or more of a signal type, a signal amplitude, a signal frequency, a signal wavelength, a signal duration, a signal code, and a signal pattern.

In certain embodiments, the determining the calibrated communication signal comprises determining at least two calibrated communication signals, the at least two calibrated communication signals differing from one another in terms of one or more of a type of signal, an amplitude of signal, a frequency of signal, a duration of signal, an harmonic in the signal, a resonance in the vibration, a rate of change of signal, and a signal pattern, a signal sequence of signal and rate of change of the sequence of vibration.

In certain embodiments, the method further comprises sending instructions to a communication device associated with the user, or to an input device associated with the user, to apply the calibrated communication signal.

In certain embodiments, the calibrated communication signal comprises one or more of an action, a spoken word, an auditive prompt, a visual prompt, a choreographic gesture, a musical tone delivered by a virtual character to be depicted on a screen of an electronic device.

In certain embodiments, the method further comprises obtaining input data of the user or the other user in response to the calibrated communication signal, and adjusting at least one parameter associated with the calibrated communication signal until the desired arousal state is achieved, the input data relating to one or more of a direct response of the user, a physiological response of the user, and observational data regarding the arousal state of the user from the other user.

In certain embodiments, the determining the calibrated communication signal comprises the processor receiving a desired communication to be communicated to the user from another user of the system, transposing the desired communication using parameters determined by the calibrated communication signal, and sending instructions to the input device or the communication device to provide transformed desired communication.

In certain embodiments, the method further comprises applying the calibrated communication signal until one or more of: the user communicates a desire to stop; a desired physiological measure of the user is detected; a predetermined time has lapsed; a fail-safe level is reached or exceeded, the user achieves self-regulation.

In certain embodiments, the method further comprises monitoring physiological data associated with the user, and when a predetermined trigger is noted, determining the required modulation and the calibrated communication signal to the user according to a predetermined desired arousal state, optionally wherein the predetermined trigger comprises a threshold value of the monitored physiological data.

From a further aspect, there is provided a system for determining a calibrated communication signal for arousal state modulation of a user, the system comprising : a computer system having a processor, the processor arranged to: obtain input of a current arousal state of the user; obtain input of a desired arousal state for the user; determine a required modulation to the current arousal state of the user to achieve the desired arousal state or to maintain the current arousal state; and determine the calibrated communication signal effective to achieve the required modulation for the user or to maintain the current arousal state.

In certain embodiments, the system further comprises a monitoring device, operably connected to the processor for providing a calibrated communication signal to the user, the calibrated communication signal being effective to modulate the user from the arousal state to the desired arousal state. The monitoring device may comprise an electronic device having a screen for displaying a virtual character (e.g. an avatar) to provide the calibrated communication signal to the user.

In certain embodiments, the system further comprises a communication device, operably connected to the processor and optionally to the monitoring device, for providing the calibrated communication signal to the user. The communication device can be a wearable device associated with the user.

In certain embodiments, the system further comprises an input device, operably connected to the processor, for obtaining input data relating to a response of the user to a communication signal, the input device comprising a wearable device has one or more sensors arranged to measure one or more of: a direct response of the user comprising one or more of a touch response, a sound response, a kinetic response, a brain signal response, a breath response, and a facial response from the user in reaction to the communication signal, and a physiological response of the user comprising one or more of a heart rate, a breathing rate, a blood flow, a sweat analysis, a measure of movement, an electrical brain signal, a temperature, a breath analysis, and one or more biomarkers of stress.

In certain embodiments, the wearable device has a screen for providing the calibrated communication signal to the user.

In certain embodiments, the system further comprises a further input device for measuring contextual data.

From another aspect, developers have identified that an arousal state of the user can be determined by determining an initial arousal state of the user, then correcting or validating the initial arousal state to obtain the arousal state of the user. Accordingly, aspects of the methods and systems of the present technology comprise determining an initial arousal state of the user, then correcting or validating the initial arousal state to obtain the arousal state of the user. The correction or validation may be determined according to the aspects and embodiments described herein.

From another aspect, there is provided a method for user arousal state regulation, the method comprising a computer system having a processor, the processor operatively communicable with: an input device associated with the user for measuring input data, the input data comprising one or more of: direct user input, physiological user input and contextual input, and a communication device for providing calibrated communication signals to the user, the processor arranged to execute a method comprising: obtaining the input data, determining the calibrated communication signal for the user based on the input data and a user profile of the user, sending instructions to the communication system to provide the calibrated communication system to the user.

In certain embodiments, the communication device has a screen for displaying a virtual character and at least some of the calibrated communication signal is delivered through the virtual character. The input device may be a wearable device.

In certain embodiments, the method comprises receiving input data relating to a preference of the user relating to the communication signal. The preference may include the user's preference relating to a personae, look, sound, and speech parameters of the virtual character.

In certain embodiments, the screen of the communication device is a touch screen and is arranged to obtain, as input data, parameters relating to the user's touch of the screen.

In certain embodiments, the method further comprises obtaining input data relating to an other user. The method comprising, in certain embodiments, taking into account the input data relating to the other user when determining the calibrated communication signal for the user. The method may comprise determining an arousal state of one or more of the user, and the other user, based on the direct user input, physiological user input and contextual input of the user and/or the other user, and optionally a user profile. In certain embodiments, the method comprises adapting the determined calibrated communication signal for the user based on the determined arousal state of the other user.

From another aspect, there is provided a system for user arousal state regulation, the system comprising a computer system having a processor, the processor operatively communicable with: an input device associated with the user for measuring input data, the input data comprising one or more of direct user input, physiological user input and contextual input, and a communication device for providing to the user calibrated communication signals determined based at least in part on the input data.

In certain embodiments, the communication device has a touch screen which is arranged to obtain input of the user's touch of the screen. The input device is a qwearable device in certain embodiments.

In certain embodiments, the system further comprises another wearable device associated with an other user for measuring input data relating to the other user, the input data relating to the other user comprising one or more of direct user input, physiological user input and contextual input.

In certain embodiments, the processor is configured to determine an arousal state of one or more of the user, and the other user, based on the direct user input, physiological user input and contextual input of the user and/or the other user, and optionally a user profile.

In certain embodiments, the processor is configured to determine a calibrated communication signal for the user based on one or more of the direct user input, physiological user input and contextual input of the user and/or the other user, and optionally a user profile.

In certain embodiments, the processor is configured to adapt the determined calibrated communication signal based on the determined arousal state of the other user.

From a yet further aspect, there is provided a wearable device comprising: at least one body portion and at least one coupling portion for coupling the wearable device to the user, at least one input unit associated with the body for obtaining input data relating to the user, at least one output unit associated with the body for providing a communication signal to the user. The wearable device may include a communication module for receiving instructions for one or more of: obtaining the input data, sending the input data to the processor of the system, providing the communication signal to the user, providing the communication signal to the other user, providing the calibrated communication signal to the user, and providing the calibrated communication signal to the other user, as described above.

In certain embodiments, the at least one input unit comprises a first sensor arranged to obtain physiological data relating to the user, and an interface for obtaining direct user input.

In certain embodiments, the interface comprises a touch screen, or buttons on a face of the body for obtaining a touch input from the user.

In certain embodiments, the at least one output unit comprises at least one actuator for providing a haptic signal to the user.

In certain embodiments, any one or more of the input unit and the output units are removeably attachable to the wearable device, for ease of replacing parts.

In certain aspects and embodiments of the methods and systems described herein, the input data includes observational input relating to the user, such as, but not limited to, a questionnaire, a diary, comments from the other user, etc.

In certain one or more of the uses of the methods and systems described herein, advantageously, the user's arousal state is determined without reliance on the user or another user (such as a caregiver, a teacher, parent, employer, etc) to identify their own arousal state.

In certain embodiments of the technology described, an improved regulation of the arousal state of the user is achieved. In the case of user's with ASD, this can help the users with academic outcomes, achievements and functionality in their lives, and increasing mortality rates.

In the context of the present specification, unless expressly provided otherwise, a computer system may refer, but is not limited to, an “electronic device”, an “operation system”, a “system”, a “computer-based system”, a “controller unit”, a “control device” and/or any combination thereof appropriate to the relevant task at hand.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives. Still in the context of the present specification, “a” computer-readable medium and “the” computer-readable medium should not be construed as being the same computer-readable medium. To the contrary, and whenever appropriate, “a” computer-readable medium and “the” computer-readable medium may also be construed as a first computer-readable medium and a second computer-readable medium.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is an illustration of a computing environment, according to embodiments of the present technology;

FIG. 2 is an illustration of an environment for implementing methods and systems of the present technology, according to embodiments of the present technology;

FIGS. 3A and 3B are illustrations of different environments for implementing methods and systems of the present technology, according to embodiments of the present technology;

FIG. 4A is a top plan view of a wearable device, according to embodiments of the present technology;

FIG. 4B is a side view of the wearable device of FIG. 4A;

FIG. 4C is a schematic representation of the wearable device of FIG. 4A;

FIG. 5 is an illustration of a monitoring device, according to embodiments of the present technology;

FIG. 6 is an illustration of a system, including various modules executed by the system, according to embodiments of the present technology;

FIG. 7 illustrates exemplary sensory profiles of a plurality of users, according to embodiments of the present technology;

FIG. 8 illustrates exemplary contextual factor profiles of a user, according to embodiments of the present technology;

FIG. 9 illustrates exemplary physiological parameter profiles of a user, according to embodiments of the present technology;

FIG. 10 illustrates method steps executed by the set-up module of FIG. 6, according to embodiments of the present technology;

FIG. 11A illustrates method steps executed by the arousal state module of FIG. 6, according to embodiments of the present technology;

FIG. 11B illustrates method steps executed by the arousal state module of FIG. 6 to determine User Index, according to embodiments of the present technology;

FIG. 12 illustrates method steps executed by the calibration module of FIG. 6, according to embodiments of the present technology;

FIG. 13 illustrates method steps executed by the arousal state module of FIG. 12, according to embodiments of the present technology;

FIG. 14 illustrates method steps executed by the modulation module of FIG. 6, according to embodiments of the present technology; and

FIG. 15 illustrates method steps executed by the monitoring module of FIG. 6, according to embodiments of the present technology.

It should be noted that, unless otherwise explicitly specified herein, the drawings are not to scale.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some embodiments of the present technology, the processor may be a general purpose processor, such as a central processing unit (CPU) or a processor dedicated to a specific purpose, such as a digital signal processor (DSP) or a Graphical Processing Unit (GPU). Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown. Moreover, it should be understood that module may include for example, but without being limitative, computer program logic, computer program instructions, software, stack, firmware, hardware circuitry or a combination thereof which provides the required capabilities.

With these fundamentals in place, we will now consider some non-limiting examples to illustrate various implementations of aspects of the present technology.

Certain aspects and embodiments of the present technology, are directed to methods and systems for, one or more of:
determining an arousal state of a user (also referred to as “current arousal state”),
determining calibrated communication signals for maintaining or changing (modulating) an arousal state of the user or for communicating with the user,
monitoring the arousal state of the user,
training a Machine Learning Algorithm to determine the arousal state, to determine the calibrated communication signal, and/or to monitor the arousal state.

Modulation of an arousal state in certain embodiments means moving the user from one arousal state to another, for actively maintaining the user in a certain arousal state, for moving the user within a given arousal state, or for extending or shortening the duration of time spent by the user in a given arousal state. Without being held to any theory, sensory modulation refers to a complex central nervous system process by which neural messages that convey information about the intensity, frequency, duration, complexity, and novelty of sensory stimuli are adjusted. Arousal states may be classified as HIGH, MID and LOW. The user may have a cognitive condition such as autism spectrum disorder or ADHD, or be neuro-atypical in other respects. In these cases, the arousal states may be relative to a crisis or a critical adverse arousal state, for example, dysregulation, overload, meltdown, epilepsia or any other epileptiform brain patterns. The user may have any other condition in which monitoring or modulation of their arousal states would be helpful for example to assist or enhance certain tasks such as learning, self-regulation, and coping mechanisms, to name a few.

### Computing Environment

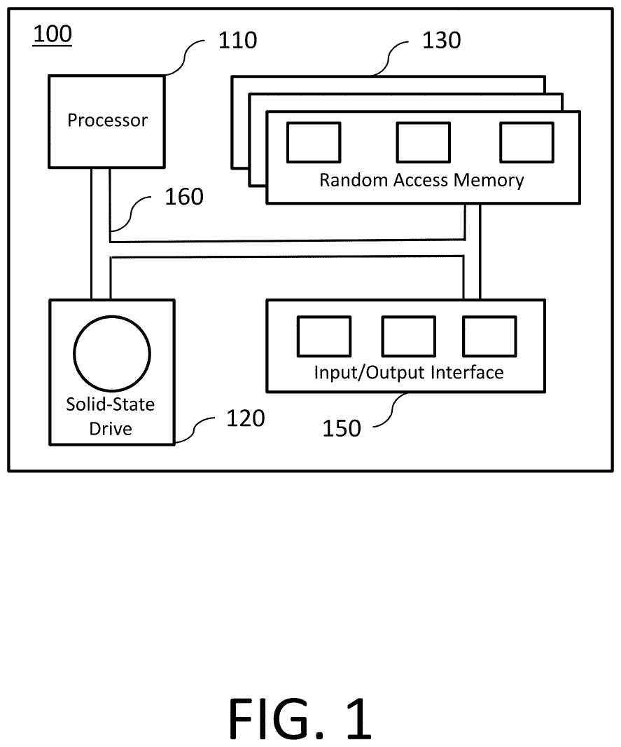

FIG. 1 illustrates a diagram of a computing environment 100 in accordance with an embodiment of the present technology. The computing environment 100 may be a computer specifically designed for calibrating communication signals. In other embodiments, the computing environment 100 may be a generic computer system.

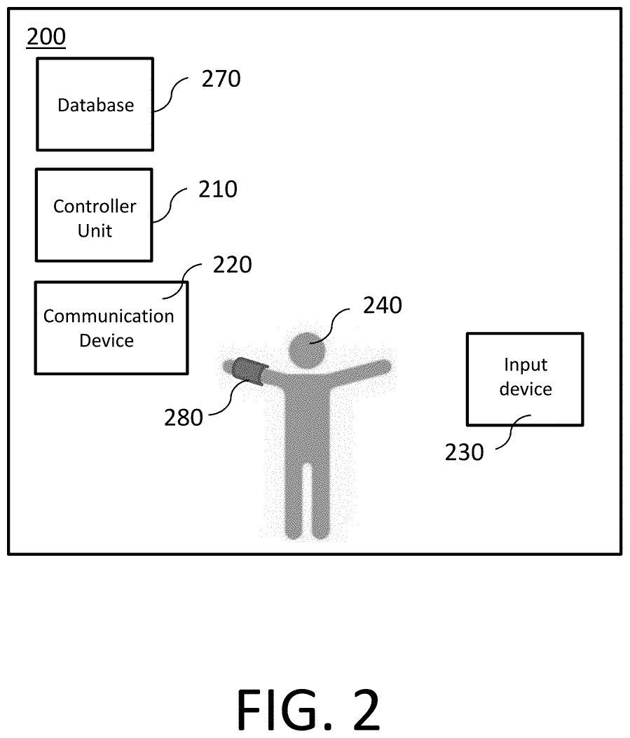

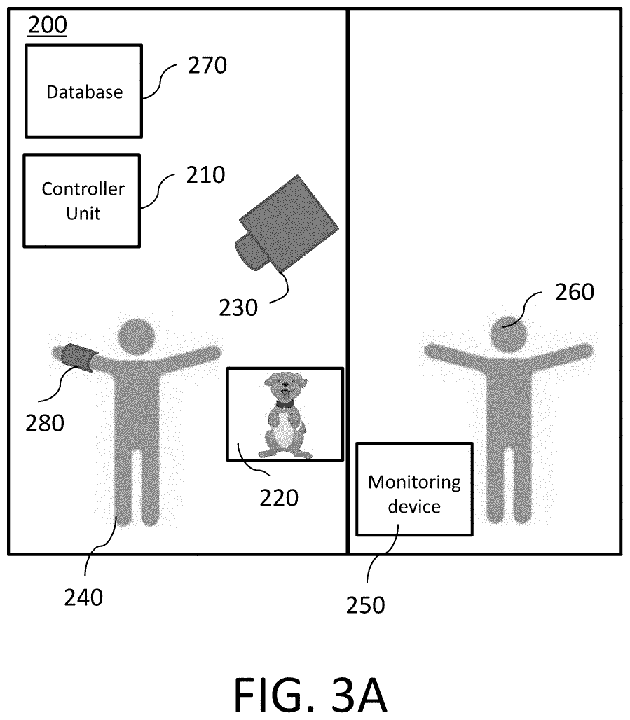

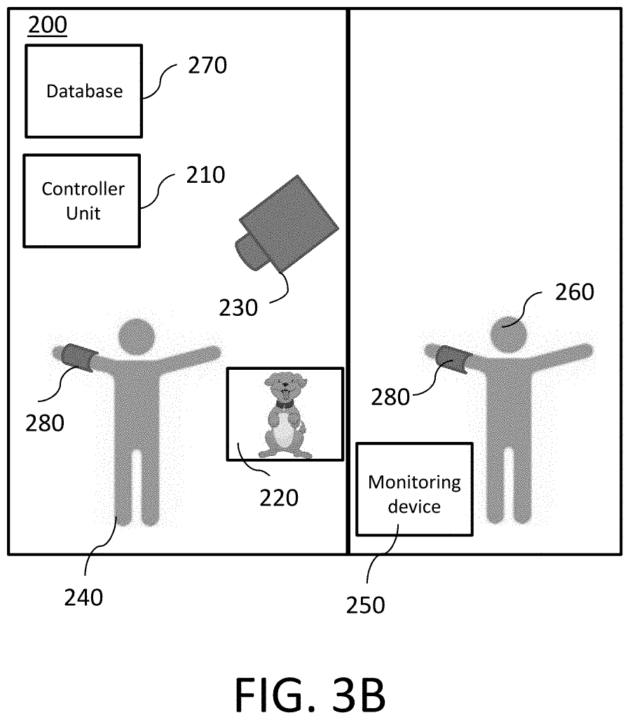

In some embodiments, the computing environment 100 may be implemented by any of (i) a conventional personal computer, (ii) a computer or a processor dedicated to operating any of a communication device 220 for providing communication signals to a user, (iii) an input device 230 for receiving input data relating to the user or input data relating to contextual information which may be relevant for assessing a current arousal state of the user, or (iv) a monitoring device 250 associated with an other user 260. The other user 260 may be any other person, such as a monitoring the user, e.g. a parent, a child, a relative, a medical practitioner, a caregiver, a therapist, a teacher etc. The other user 260 may be a friend or another requiring arousal state monitoring. Any of the communication device 220, the input device 230 or the monitoring device 250 can be an electronic device such as, but not limited to, a laptop, a mobile device, a tablet device, one or more wearable devices such as a bracelet, a watch, a strap-on device to be worn on any one or more of the arms, legs, chest etc. Embodiments of the communication device 220, the input device 230 and the monitoring device 250 will be described more fully below with reference to FIGS. 2 and 3.

In some embodiments, the computing environment 100 may also be a sub-system of one of the above-listed systems. In some other embodiments, the computing environment 100 may be an “off the shelf” generic computer system. In some embodiments, the computing environment 100 may be distributed amongst multiple systems. The computing environment 100 may be specifically dedicated to the implementation of the present technology. As a person in the art of the present technology may appreciate, multiple variations as to how the computing environment 100 is implemented may be envisioned without departing from the scope of the present technology.

In some embodiments, the computing environment 100 comprises various hardware components including one or more single or multi-core processors collectively represented by a processor 110, a solid-state drive 120, a random access memory 130 and an input/output interface 150. Communication between the various components of the computing environment 100 may be enabled by one or more internal and/or external buses 160 (e.g. a PCI bus, universal serial bus, IEEE 1394 “Firewire” bus, SCSI bus, Serial-ATA bus, ARINC bus, etc.), to which the various hardware components are electronically coupled.

The input/output interface 150 may allow enabling networking capabilities such as wire or wireless access. As an example, the input/output interface 150 may comprise a networking interface such as, but not limited to, a network port, a network socket, a network interface controller and the like. Multiple examples of how the networking interface may be implemented will become apparent to the person skilled in the art of the present technology. For example, but without being limitative, the networking interface may implement specific physical layer and data link layer standard such as Ethernet, Fibre Channel, Wi-Fi or Token Ring. The specific physical layer and the data link layer may provide a base for a full network protocol stack, allowing communication among small groups of computers on the same local area network (LAN) and large-scale network communications through routable protocols, such as (IP).

According to implementations of the present technology, the solid-state drive 120 stores program instructions suitable for being loaded into the random access memory 130 and executed by the processor 110 for executing one or more methods, such as methods for determining a calibrated communication signals, determining a communication signal, monitoring the arousal state of the user, etc. For example, the program instructions may be part of a library or an application.

### Environment

Referring to FIG. 2, there is shown one embodiment of an environment 200 in which embodiments of the present technology may be implemented. The environment 200 is any setting in which a communication or interaction is required with the user 240 through a sensory input. The communication may be for the purposes of modulating the arousal state of the user 240 through a calibrated communication signal, for communicating with the user 240 through a calibrated communication signal, or for monitoring the arousal state of the user 240.

The environment 200 may be a home, a hospital, a clinic, a laboratory, a school, a clinical trial setting, or any other setting. The user 240 can be a neuro-atypical person, such as a person with autism for example. In other embodiments, the user 240 is another type of neuro-atypical, or is neuro-typical. For example, the environment 200 may be a setting in which a current arousal state of the user 240 is being monitored, such as a clinical trial setting or other pharmaceutical testing environment, in which an effect of a drug on user's arousal states is being evaluated.

In certain non-limiting embodiments, as illustrated in FIG. 2, the environment 200 comprises a controller unit 210 operably connectable to a database 270, one or more communication devices 220 for providing a communication signal to the user 240, and one or more input devices 230 associated with user 240 for obtaining input data from the user 240. The controller unit 210 is operated by the processor 110, in certain embodiments. In certain other embodiments, the controller unit 210 is the processor 110.

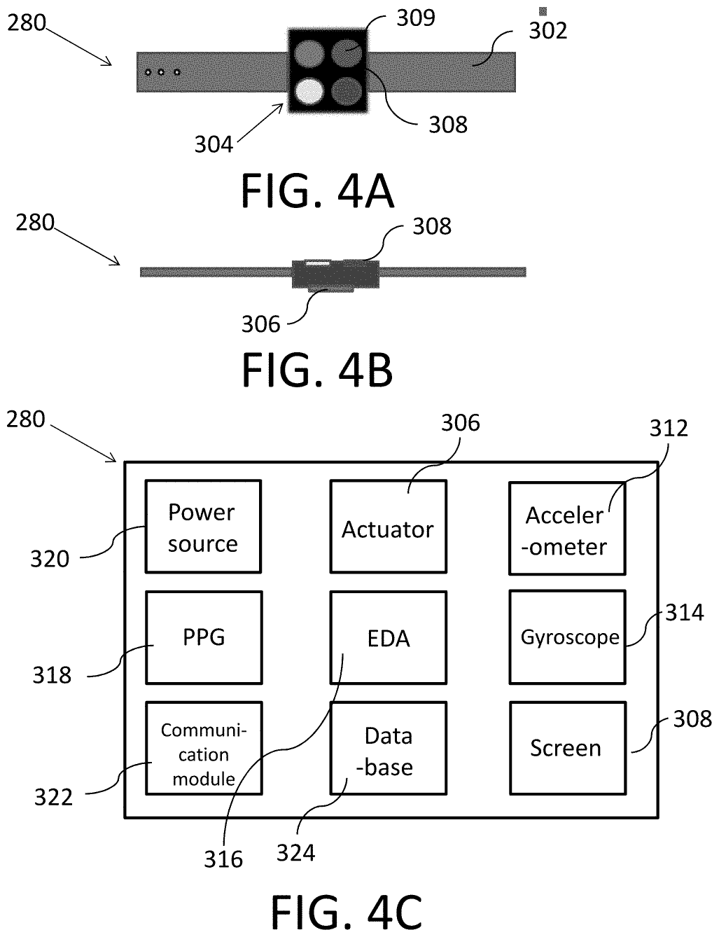

A wearable device 280, operable as both the input device 230 and the communication device 230 is also provided. The wearable device 280 will be described in more detail below with reference to FIGS. 4A, 4B and 4C. Any one or more of the input device 230, the communication device 220, and the wearable device 280 may be operably communicable with one another and/or to the controller unit 210.

The environments 200 of FIGS. 3A and 3B differ from that of FIG. 2 in that they also include one or more monitoring devices 250 associated with the other user 260. The other user 260 may be physically separated from the user 240 such as in a different location to the user 240, for remote monitoring or therapy, for example. The user 240 may be any person requiring arousal state modulation or monitoring. The other user 260 may be any other person monitoring the user 240, such as a parent, a child, a relative, a medical practitioner, a caregiver, a therapist, a teacher etc. In the environment of FIG. 3A, a camera is provided as one of the input devices 230. In FIG. 3B, wearable devices 280 are provided for both the user 240 and the other user 260.

### Communication Devices, Communication Signals and Calibrated Communication Signals

Communication devices 240 used in aspects and embodiments of the present technology comprise any device capable of providing communication signals and/or calibrated communication signals to the user in a controllable manner Communication devices 240 are also referred to herein as “stimuli devices”. Communication signals and/or calibrated communication signals generated by communication devices may convey information to the user 240, cause a reaction in the user 240, assist the user 240 to self-regulate or co-regulate, and/or be used to interact with the user 240 in any other way. In certain embodiments, the communication signal and/or calibrated communication signals is tailored to obtain a specific reaction from the user 240 or to convey specific information, for example, to help the user conduct a specific task such as studying, sports, and daily activities.

Communication signals and calibrated communication signals can be electromagnetic-based signals such as light and heat. Communication signals and calibrated communication signals can be signals which can be defined by their frequency or wavelength such as forces (e.g. haptic signals), movement (e.g. haptic signals), sounds (e.g. audio/acoustic signals), colour (e.g. visual signals including colour, brightness etc), smells (e.g. olfactory signals), telekinetic signals, electrical signals, magnetic signals, pressure signals (piezometric) etc. Electrical signals can be electrical impulses delivered to the user 240 such as through Vagus nerve stimulation. Visual signals include augmented reality images and/or virtual reality images in certain embodiments.

Haptic signals include forces, vibrations or motions generated in any way such as using an actuator which contacts the user 240, or using a “contactless” haptic actuator arranged to deliver ultrasound waves, air pressure or the like to create acoustic or air pressure. Haptic signals generated in any other way, and using any type of actuator, are also included within the scope of the present technology.

In certain embodiments, one or both of the communication signal and the calibrated communication signal is defined in terms of one or more communication signal parameters such as, but not limited to, signal amplitude, signal wavelength, signal frequency, signal duration, signal sequence, signal code, and the like. By signal code is meant a combination of different signal types, such as visual, auditive, haptic, olfactive, etc.

In non-limiting embodiments, communication signals have frequencies of any range, such as acoustic, subacoustic, infracoustic, or supersonic. Combinations of sound communication signals having frequencies in different ranges are also possible. For example, communication signals which are haptic signals can have frequencies of any range, such as those produced using piezo actuators (1-500 Hz). Communication signals can also include vibracoustic signals, having a range of about 30 Hz to about 120 Hz.

Communication signals and/or calibrated communication signals can include sound, visual or haptic signals delivered by a virtual character on a screen of an electronic device such as by talking, singing, humming, moving, dancing, gesticulating etc. The virtual character (also referred to herein as “avatar”, “virtual companion” or “virtual personae”) can have any form such as human, animal or object. In this respect, the associated communication device for providing the communication signals or the calibrated communication signals to the user has a screen for displaying the virtual character, optionally speakers for providing the accompanying sound signal, and optionally actuators or other haptic mechanism for delivering the haptic signal. The communication device for delivering haptic signals can include any type of actuator, such as linear resonant actuators, piezo actuators, or rotating motors.

The screen of the communication may also be a touch screen to allow two-way communication with the virtual character. The screen could be textured. The screen may comprise a 3D surface rendering of the virtual character. The screen could be embodied in any type of device such as a virtual reality glasses, flight simulation screens, eye glasses, teletherapy screens, and lenses.

In the embodiments of FIGS. 3A and 3B, the communication device 220 comprises a screen for presenting visual images of the virtual character (avatar) with controllable visual and audio signals as sensory inputs (as the communication signal or the calibrated communication signal). The wearable device 280 implements both communication and input devices 220, 230. The screen could be a touch screen allowing two-way interaction of the user with the virtual character. It should be understood that one or more of the communication devices 220 may provide a single type of communication signal or more than one type of communication signal. In other embodiments, the communication device 220 comprises any device capable of delivering a communication signal to the user 240 such as, but not limited to, speakers, lights, robots, humanoids, avatars, holograms, screens etc.

Communication signals and/or calibrated communication signals can also be delivered to the user 240 by the other user 260, another person, or a robot.

In non-limiting embodiments, a function of the calibrated communication signal is to trigger awareness to the user 240 of an oncoming crisis or undesirable arousal state, to predict the undesirable arousal state or to avoid the undesirable arousal state. Examples of effective calibrated signals include a haptic signal emulating the pattern and frequency of the user's heart beat in cases where increased, or rapidly changing heart beat is a physiological biomarker of the undesirable arousal state. Similarly, such a calibrated communication signal emulating the user's heart beat may be in the form of any type of signal such as a sound signal, a light signal, or combinations of any of the above.

The communication device 220 may be an electrical or electro-mechanical device with simple output or may be a more complex computing entity. The communication signal can be a single, or a plurality of different types of communication signal coming from the same or different communication device.

The communication devices 220 may comprise a communication module (not shown) allowing receiving of instructions or commands for applying and/or controlling communication signal parameters to generate the desired output (e.g., a certain range, value, increase or decrease in any one or more of the amplitude, wavelength, frequency, duration, sequence, of the communication signal). In some embodiments, the instructions are received from the controller unit 210 (which can also be the processor 110) and comprise command values. In the case of the communication device 220 being a haptic device, calibration may be required to take into account the distance of the haptic actuator from the user's skin in order to deliver a desired haptic output to the user 240.

In some embodiments, each one of the one or more communication devices 220 may be commanded independently, in accordance with dedicated control values. For communication devices 220 having more than one type of signal communication capability, each type of signal can be commanded independently. For example, but without being limiting, control values may comprise a Boolean value (haptic signal_ON, haptic signal_OFF), a numerical value (Haptic signal acceleration=+/−0.5 g−1.9 g, frequency=0-1000 Hz, or 150-300 Hz. In one non-limiting embodiment, haptic signal acceleration is 1.6 g, and the haptic signal frequency is 205 Hz).

### Input Devices and Input Data

Input devices 230 used in aspects and embodiments of the present technology are adapted to measure or obtain input data associated with the user 240, as well as other input data relevant to an arousal state of the user 240. Input data includes, for example, one or more of direct input data of the user 240, indirect input data of the user 240, and contextual data relating to contextual factors relating to the arousal state of the user 240. Input data relating to the direct response of the user 240 comprises at least one measured direct response value. Input data relating to the indirect response of the user 240 comprises at least one measured indirect response value. Input data relating to the contextual response of the user 240 comprises at least one measured contextual value. Input data includes both measured input data values, as well as subsequently processed input values (such as after natural language processing, facial recognition processing, and the like).

### Direct Input Data

Direct input data of the user can be representative of a direct response of the user 240 to the communication signal, such as an interaction of the user 240 with the input device 230. The direct input data may include touch signal, a voice signal, a body movement signal (e.g. arms, body, head, eyes) from the user 240. Movement can be voluntary (such as foot tapping, clapping etc), or involuntary (such as flapping). In certain embodiments, direct input data can be considered as a voluntary or conscious user input in reaction to the communication signal.

Direct input data may include sign language, and in this respect, the input device 230 may include sign language detection capabilities, such as a camera with shape detection capabilities.

Direct input data may include spoken or mouthed words of the user 240, or sounds of the user 240. In this respect, input devices 230 may include a microphone for detecting the audio data of the user 240 for subsequent natural language processing by the input device 230 or the processor 110.

Direct input data may also include the user's responses to questions such as in a questionnaire-type format. The questionnaire may include a plurality of questions with weightings for each question or combinations of questions. In certain embodiments, the questionnaire includes custom markers for a given task of the user 240.

### Indirect Input Data

Indirect input data can be representative of an indirect response of the user 240 to the communication signal, such as a measured response or reaction of the user 240. Indirect input data can be considered as involuntary or subconscious user inputs in reaction to the communication signal. The indirect input data include, but are not limited to, one or more physiological parameters such as body temperature, heart rate, breathing (respiration) rate, movement, sweat levels, biomarkers of stress in the sweat, glucose levels, facial expressions, pupil size, electrical signals such as from the brain or eyes measured by electroencephalograms (EEG), etc. and changes in the foregoing. The physiological parameters may be any indicator or biomarker of different user arousal states, or different emotions, such as stress, hyperactivity, hypoactivity, etc. The physiological parameters may be any indicator or biomarker of a change in arousal state of the user, such as an indicator or precursor of a dysregulation event such as a meltdown or crisis state. For example, in certain embodiments, biomarkers of a change in arousal state comprise an increase in heart rate, an increase in temperature, body localisation in space (“flapping”), proprioception, time-referencing behaviour or geo-referencing. Other biomarkers of stress or arousal state may include changes in sleep patterns, behavioural patterns, language style, vocabulary style, competency in known tasks, time referencing, and the like. Baselines of all biomarkers are established in a set-up phase for the user 240.

Other input data may be representative of “avoidance” or “flight” type responses of the user 240 to the communication signal. They can be captured manually by the other user 260 or an observer, or automatically by the input device 230 through suitable sensors such as geolocation etc. The input data for “avoidance” or “flight” can also be determined by the user 240, or the input device 230 after the fact, for example by voluntary removal of the input device 230.

### Contextual Data

Contextual data includes contextual factors and their associated values that may affect the user's arousal state. Contextual data may include stressors for the user 240. Contextual factors may include, for example, environmental conditions. Contextual data may comprise data relating to the environment such as temperature data, atmospheric data, visual data, light frequency submission (e.g. incandescent light, blue dominant spectrums, etc), audio data, data on levels of air quality (e.g. pollution levels) etc. Contextual data may also include data regarding tiredness levels, food intake (as the user may have sensitivity, intolerance or allergies to certain foods), medication etc. Contextual data may be provided by the input device 230 or by other means such as from databases or lookup tables. Contextual data may also be provided by someone other than the user, for example the other user 260, on comments and observations on the user.

### Input Devices

Input devices 230 for measuring the input data comprise sensors for detecting and measuring the input data. The sensors can be any type of sensor for detecting any type of input data, whether direct, indirect or contextual.

Example input devices 230 for detecting and measuring direct input data include those having, for example, one or more of: a user interface such as a touch screen or a keyboard/mouse for touch input data, a microphone for sound input, an accelerometer for movement input, an object to be thrown or manipulated, and the like. Examples of such input devices 230 include a portable device such as the wearable device 280, a tablet, a mobile telephone or a laptop.

Non-limiting examples of input devices 230 for measuring or detecting indirect input data include detection devices or any other type of device including one or more sensors, such as a thermometer, a gyroscope, electrocardiogram sensor, a sweat sensor, an ophthalmoscope, a sphygmomanometer, EEG or a camera. In some embodiments, the input device 230 is the wearable device 280.

Non-limiting examples of input devices 230 that detect and measure contextual data include those including sensors for monitoring the environment such as an air temperature thermometer, an ultra-violet (UV) sensor, an atmospheric humidity sensor, an atmospheric pressure sensor, a CO₂ sensor, an O₂ sensor, a gas composition sensor, a light level sensor, a colour sensor (e.g. a spectrometer), a polarimeter for measuring polarisation of the light, a microphone. Alternatively, environmental conditions may be obtained from databases, such as meteorological databases.

In non-limiting embodiments, input devices 230 are arranged to measure and detect one or more types of data (direct input data, indirect input data or contextual data). In certain embodiments, the input device 230 comprises a plurality of wearable devices 280, each one of the plurality of the wearable devices 280 arranged to measure the same or different parameters.

In the embodiment of FIG. 3A, one of the input devices 230 is an image capturing device 230, such as a video camera. In some embodiments, the video camera is configured to capture images and/or videos of the user's face and/or of an object surrounding the user. This image data can be converted to another form of data through face recognition software for example. In certain embodiments, the input device 230, or a processing system associated with the input device 230, can detect and process sign language of the user 240.

In certain embodiments, the input device 230 includes a microphone for detecting speech and sounds from the user 240, which may undergo subsequent natural language processing. This has uses in cases where the user 240 has a condition affecting their speech, such as Gilles dela Tourette syndrome.

In certain embodiments, the input device 230 comprises an interface for receiving direct user input in the form of a questionnaire. The questionnaire may include a plurality of questions with weightings for each question or combinations of questions. In certain embodiments, the questionnaire includes custom markers for a given task of the user 240.

In non-limiting embodiments, the input devices 230 are configured to transmit the input data, whether it is direct input data, indirect input data, or contextual data. In this regard, the input devices 230 may comprise a communication module (not shown) for transmitting the input data to the controller unit 210. In some embodiments, the connection between each of the input devices 230 and the controller unit 210 is wired. In some other embodiments, the connection between the input devices 230 and the controller unit 210 is wireless.

In non-limiting embodiments, a plurality of input devices 230 are provided, each being associated with a respective user. Input data from each of the input devices 230 is arranged to be sent between the users, for example for the purposes of co-regulation.

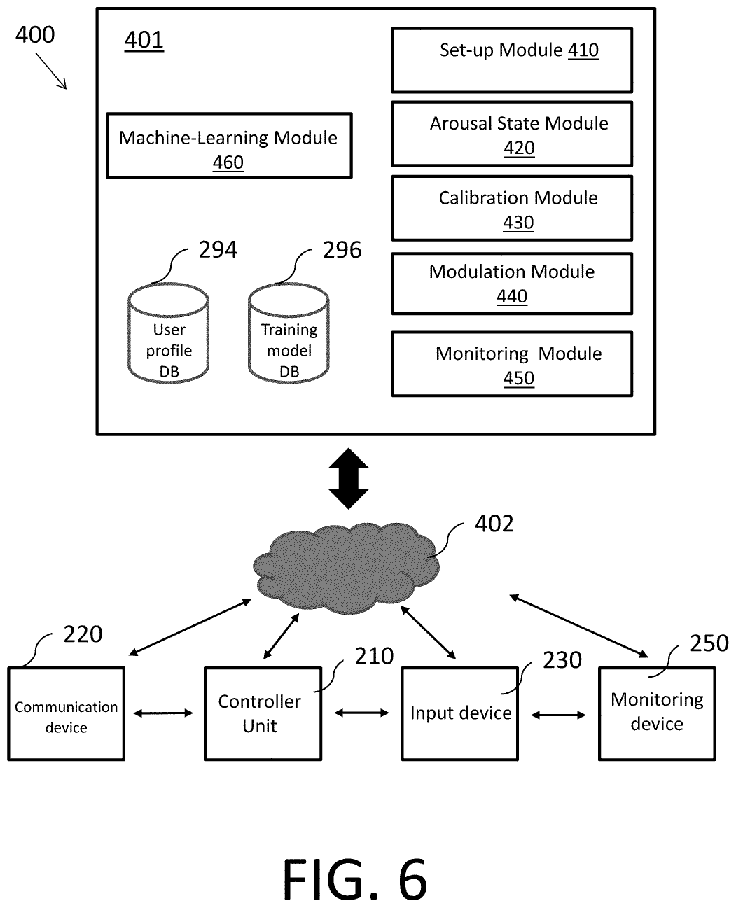

In some embodiments, the input device 230 is arranged to the send the input data to the controller unit 210 for storage in a database 290. The input data may be used as an input to training a machine learning algorithm. The database 290 may comprise one or a plurality of separate databases for storing various datasets and/or algorithms Without limitation, the database 290 is arranged to store any one or more of (i) input data received from one or more of the input devices, (ii) user profile data, (iii) instructions for determining a current arousal state of the user, (iv) determined current arousal state of the user, (v) instructions for determining a calibrated communication signal, (vi) a determined calibrated communication signal, and (vii) instructions for applying calibrated communication signals. In certain embodiments (FIG. 6), the database comprises a user profile database 294 and a training model database 296.

Any one or more of the communication device 220 and the input device 230 can be incorporated into a single device or be separate devices in any combination thereof appropriate to the relevant task at hand. For example, the input devices 230 may be included within one or more devices. So a single input device 230 can have a number of different sensor functions. If a plurality of input devices 230 are provided, each one of the input devices 230 can have the same or different sensor functions. For example, the wearable device 280 has sensors for monitoring any one or more of heart rate, blood pressure, pupil dilation, electrodermal activity, and movement, and a touch screen. The wearable device 280 also implements various communication devices 220 for providing communication signals to the user such as a screen, a speaker and a haptic actuator (whether contact or contactless). In some embodiments, the monitoring device 250 and the input device 230 may be embodied in a single device permitting the inputting of contextual data by the other user 260, as well as monitoring various input data associated with the user 240. In some embodiments, more than one wearable device 280 may be provided incorporating different sensors, such as one wearable device 280 being wearable on the wrist sensing heart beat, and another wearable device being wearable on another part of the body for sensing sweat factors. In certain embodiments, the wearable device 280 is worn on the leg.

Furthermore, both the user 240 and the other 260 may have input devices 230 associated with them, with the input data from both being used to determine the communication signal or the calibrated communication signal.

### Monitoring Devices

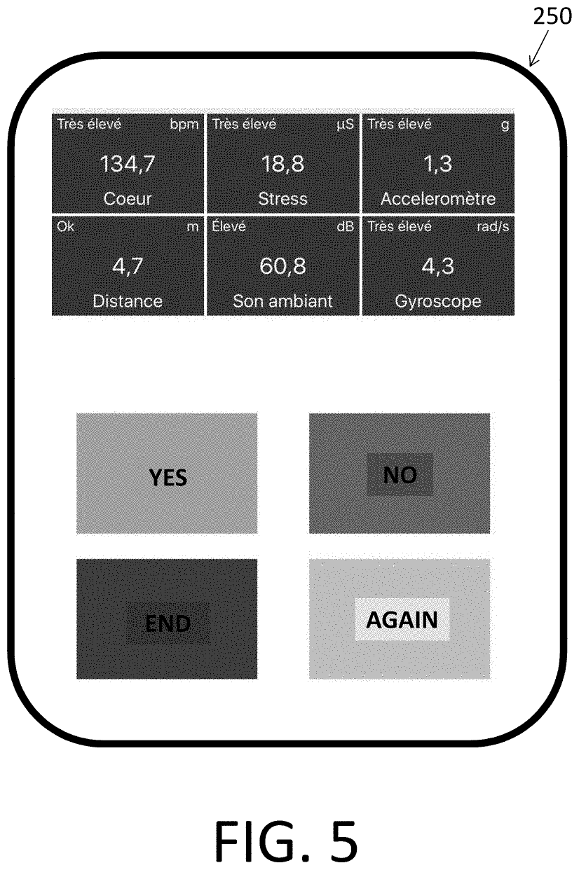

Monitoring devices 250 can be any device associated with the other user 260 for monitoring one or more of: the user 240, the communication device 220, the communication signals, the input device 230, and/or the input data. Monitoring devices 250 include, but are not limited to, cameras, microphones, tablets, mobile devices, personal computers. In certain embodiments, the monitoring devices 250 permit input from the other user 260, which may contribute to control of the communication devices 220 associated with the user 240. In one embodiment, the monitoring device 250 may permit contextual data input or other type of input, such as the responses to at least part of the questionnaire, by the other user 260. Monitoring devices may include one or more sensors. One embodiment of a monitoring device 250 in the form of a tablet device will be described below with reference to FIG. 5.

Other examples of communication devices 220, communication signals, input devices 230, input data, monitoring devices 250 and/or monitoring data may also be envisioned without departing from the scope of the present technology.

### Controller Unit

In some non-limiting embodiments, the controller unit 210 is connected to the one or more of the communication devices 220, the one or more of the input devices 230 and/or the one or more of the monitoring devices 250. The connection may be wired or wireless. In some embodiments, the controller unit 210 may be implemented in a similar way as the computing environment 100 and may comprise control logic to control the one or more of the communication devices 220, the one or more of the input devices 230 and/or the one or more of the monitoring devices 250. In some non-limiting embodiments, the controller unit 210 is the same as the processor 110. In some embodiments, the controller unit 210 may receive data from and/or transmit data to the one or more of the communication devices 220, the one or more of the input devices 230 and/or the one or more of the monitoring devices 250.

In some other non-limiting embodiments, functions of the controller unit 210 may be distributed across the one or more of the communication devices 220, the one or more of the input devices 230 and/or the one or more of the monitoring devices thereby resulting in a configuration wherein the one or more of the communication devices 220, the one or more of the input devices 230 and/or the one or more of the monitoring devices 250 comprises control logic. In such embodiment, the controller unit 210 as a standalone unit may not be required.

### Wearable Devices as Input and Communication Devices

As mentioned above, input devices 230 and communication devices 220 can be in the form of a wearable device. FIGS. 4A, 4B and 4C illustrate one embodiment of the wearable device 280. The wearable device 280 is adapted to be worn around the wrist of the user 240, like a bracelet or a watch. In other embodiments, the wearable device 280 can be adapted to be worn on any part of a user's body, such as the ankle. In yet other embodiments, the wearable device 280 is adapted to be worn across the chest of the user 260. The wearable device 280 comprises a coupling portion 302 for coupling to the user's body part and a body portion 304 including one or more input units and one or more output units.

In non-limiting embodiments, the coupling portion 302 is a strap for attaching the wearable device 280 to the wrist of the user. The strap is adjustable to adjust the proximity of the body portion 304 to the user's skin. In other embodiments, the wearable device 280 may be incorporated into an item of clothing. In other embodiments, the wearable device 280 may comprise only a body portion 304, or only a coupling portion.

The wearable device 280 is sized, shaped and otherwise configured to be comfortable to the user. For example, the parts of the coupling portion 302 and/or body portion 304 may be sized and shaped for comfort. Parts of the coupling portion 302 may be lined with a soft material for additional comfort. At least a part of the wearable device 280, such as those portions in contact with the user's skin or other body parts, is made of a material which does not irritate the user's skin or other body part.

The body portion 304 has a housing that incorporates a number of input units and output units. The input units comprise the input devices 230 or sensors for obtaining input data of the user 240, and the output units comprise the communication devices 220 described above. As shown in FIG. 4C, one or more actuators 306 are provided extending from the body portion 304 for generating haptic signals; a screen 308 is provided for presenting visual signals and allowing direct user input through at least touch; a speaker 310 is provided for presenting audio signals; and various sensors are provided for obtaining input data. The sensors include, in this non-limiting embodiment, an accelerometer 312 for measuring linear movement, a gyroscope 314 for measuring angular orientation, an electrodermal (EDA) sensor 316 for measuring sweat levels, and a PPG (photoplethysmography) sensor 318 for sensing the rate of blood flow.

The wearable device 280 may include any other sensors for detecting and measuring the input data. For example, the wearable device 280 may also include sensors for obtaining contextual data about the environment, such as but not limited to: barometric pressure, relative humidity, ambient temperature, light levels and spectrum, etc. The wearable device 280 may also include a microphone for detecting sound and speech of the user 240 for undergoing natural language processing. In other embodiments, the wearable device 280 includes an electrocardiogram (ECG) sensor 318 for measuring the heart rate. The wearable device 280 may include any other output units for providing the communication signal or the calibrated communication signal to the user 240.

In certain embodiments, the wearable device 280 also includes one or more of a power source 320, a communication module 322 for sending and receiving data and commands between the wearable device 280 and the controller unit 210, and a database 324 for storing input data or commands The database 324 can be a flash storage unit or any other type of storage unit. In non-limiting embodiments, the various components housed within the body of the wearable device 280 are positioned and connected relative to each other such that they are easily accessible and replaceable.

The interactive screen 308, best seen in FIG.4A, comprises four (4) colour indicators 309 which the user 240 can interact with in response to the communication signal. The indicators 309 can represent a communication from the user 240 to “stop” (blue indicator), “yes” (green indicator), “no” (the red indicator), “again” (the yellow indicator). It will be appreciated that more or less than the four indicators 309 can be provided, that the indicators 309 may have none or different labelling than those indicated, and that the indicators 309 can be representative of any other communication from the user 240. The indicators 309 are capacitive buttons, switches or any other touch sensitive technology. Pushing of the indicators may initiate a confirmation vibration from the haptic actuator 306.

Instead of an interactive screen 308, the body portion 304 may include interactive components such as buttons, keys, switches etc. In other embodiments (not shown), the wearable device 280 includes a plurality of user input buttons on a face of the body portion 304. The user input buttons may have the same or different marks as the indicators 309, such as colours, letters, marks, textures etc.

In non-limiting embodiments, the interactive screen 308 is adapted to issue alerts or other communications to the user 240. In non-limiting embodiments, the interactive screen 308 is adapted to display the virtual character delivering the calibrated communication signals or the communication signals. Example alerts could be for medication adherence.

In certain embodiments (as illustrated in FIG. 3B), both the user 240 and the other user 260 are arranged to each wear a wearable device 280, the system 400 being arranged to allow one or both of two-way communication between the user 240 and the other user 260 through the respective wearable devices, and co-regulation of the respective arousal states of user 240 and the other user 260.

### Tablet Devices as Monitoring Devices and Communication Devices

FIG. 5 depicts an embodiment of the monitoring device 250, which can also function as the communication device 220, implemented here as an electronic device, such as a tablet, having a display screen 350. The monitoring device 250 and/or the communication device 220 has a communication module (not shown) which is arranged to receive the input data from the wearable device 280 and present it on the display screen 350 to allow the other user 260 to monitor the measured physiological data, amongst other data, of the user 240. Also presented on the display screen 350 is the input data from the user 240 via the indicators of the wearable device in response to the communication signal.

In other embodiments (not shown), one or both of the monitoring device 250 and the communication device 220 also provide an input interface for the other user 260 to provide certain set-up or calibration input such as a contextual factor profile of the user 240, contextual data, a sensory profile of the user 240, a current arousal state of the user 240, a desired arousal state of the user 240, a validation of a current arousal state of the user 240, a validation of a proposed communication signal, and predetermined threshold parameters. Observations of the other user 260 about the user 240 can be input as input data through the monitoring device 250.

In other embodiments (not shown), the monitoring device 250 is arranged to display the virtual character (for example, as a depicted in FIG. 3 as a dog) for providing the communication signal or the calibrated communication signal to the other user 260.

The monitoring device 250 may also be arranged to receive instructions from the other user 260 regarding the calibrated communication signal, which instructions are transformed to the calibrated communication signal. For example, the other user 260 may instruct the monitoring device 250 to send a calibrated communication signal to the user 240 with a message of “Time to study mathematics”. This message will be transformed and delivered to the user 240, by the virtual character speaking, on the communication device 220 with a voice speed, voice timbre, speech speed determined as the calibrated communication signal. The determination of the calibrated communication signal may also take into account the detected arousal state or other input data from the other user 260.

### Systems

Non-limiting aspects and embodiments of the present technology comprise systems 400 for one or more of: determining an arousal state of the user 240, determining a calibrated communication signal for the user 240, monitoring an arousal state of the user 240, and training an MLA to determine arousal states or calibrated communication signals. One embodiment of such a system 400 is shown in FIG. 6.

As depicted, in non-limiting embodiments, the system 400 comprises a computer system 401 operatively communicable with one or more of: the communication device 220, the input device 230, the monitoring devices 250, and the controller unit 210, via a communication channel 402.

In certain embodiments, the computer system 401 implements the computing environment 100. In certain embodiments, the computer system 401 includes the controller unit 210, or is implemented with the controller unit 210.

In some embodiments, the communication channel 402 is the Internet and/or an Intranet. Multiple embodiments of the communication channel 402 may be envisioned and will become apparent to the person skilled in the art of the present technology.

In some embodiments, the computer system 401 may be connected to the communication devices 220 and/or the input devices 230 via the controller unit 210. In some other embodiments, the computer system 401 may be directly connected to the communication devices 220, the input devices 230 and/or the monitoring devices 250. In some alternative embodiments, the computer system 401 is implemented, at least partially, on the controller device 210. In yet some alternative embodiments, the computer system 401 may be distributed across the controller unit 210, the communication devices 220, the input device 230 and/or the monitoring devices 250.

In some embodiments, the computer system 401 may be implemented on a computing environment similar to the computing environment 100. In some embodiments, the computer system 401 may be hosted on a server installed within or in a vicinity of the environment 200. In some alternative embodiments, the system 400 may be partially or totally virtualized through a cloud architecture.

In some embodiments, the computer system 401 comprises a set-up module 410, an arousal state module 420, a calibration module 430, a modulation module 440, and a monitoring module 450. In some embodiments, the computer system 401 also comprises a machine-learning module 460. Any one or more of the set-up module 410, the arousal state module 420, the calibration module 430, the modulation module 440, the monitoring module 450 or the machine-learning module 460 may access the user profile database 294 and/or the training model database 296.

### The Set-Up Module and User Profiles

In certain embodiments, the set-up module 410 is configured to collect data or information relating to the user 240, generally referred to herein as “user profiles”. User profiles include any information and data relating to the user including parameters affecting or defining a current arousal state of the user. The parameters affecting or defining the current arousal state can be environmental, or physiological, for example. The data and information obtained by the set-up module 410 may then be used by the computer system 401 to initiate an execution of one or more methods for determining a current arousal state of the user 240, determining a calibrated communication signal for the user 240, and for monitoring a current arousal state of the user 240 or for monitoring input data.

User profiles, obtained or managed by the set-up module 410, comprise but are not limited to: a sensory profile, a contextual factor profile, a physiological parameter profile, a disorder profile, and an interaction profile. The user profiles include data relating to parameters in the given profile, and relationships between the parameters. In certain embodiments, at least some of the user profiles define baselines of certain of the parameters. At least some of the user profiles and related parameters of the set-up module 410 are further elaborated on below:

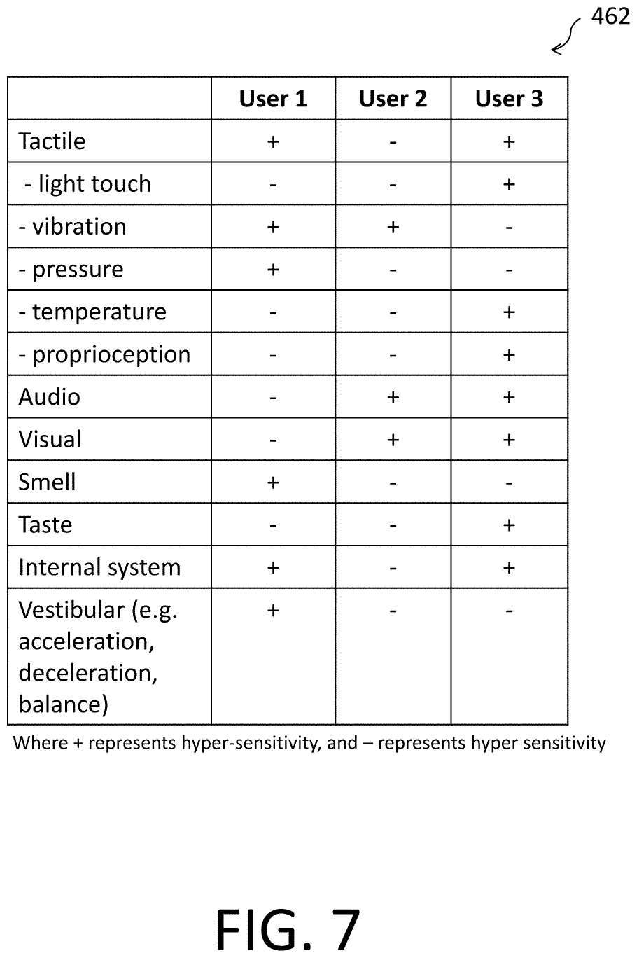

(i) Sensory profile. The sensory profile 462 defines the user's senses, such as the sensitivity of the user's senses. As can be seen in an example shown in FIG. 7, the sensory profile 462 for the user 240 includes information on sensory processing patterns for the user (e.g. hypo-reactive or hyper-reactive). Sensory profile data can be collected for a plurality of users and stored together or separately. The sensory profile 462 can include sub-categories, such as those of the tactile sense as shown in FIG. 7. The sensory profile 462 relates to inherent or genetic dispositions of the sense sensitivity. This data can be collected and stored in the user profile database 294, or across a number of different databases. The sensory profile 462 can include genetic biomarkers. The sensory profile may include information regarding the verbal level of the user 240 (e.g. verbal, non-verbal) and its affect on the senses of the user 240.

(ii) Contextual factor profile. The contextual factor profile of the user 240 defines parameters relating to the impact of various contextual factors on one or both of the sensory profile of the user 240 and the arousal state of the user 240. Contextual factors can include various external factors that may affect the sensitivity of one or more senses of the user 240, and/or a default arousal state of the user 240. Contextual factors include, but are not limited to, a tiredness level of the user 240; environmental factors or stressors such as ambient noise, pollution, humidity and temperature; medication being taken by the user and how it impacts the sense sensitivity of the user 240 including sensitivity fluctuations during a dose interval (e.g. the user may become more sensitive to sound towards the end of a dose interval); the brightness of the environment; the people in the environment; interaction with a person or people in the environment (e.g. a caregiver, a teacher etc); the number of people in the environment; food intolerances, sensitivities and allergies. The contextual factor profile can be predetermined. Contextual factor profile includes contextual data and contextual factor weights in certain embodiments.

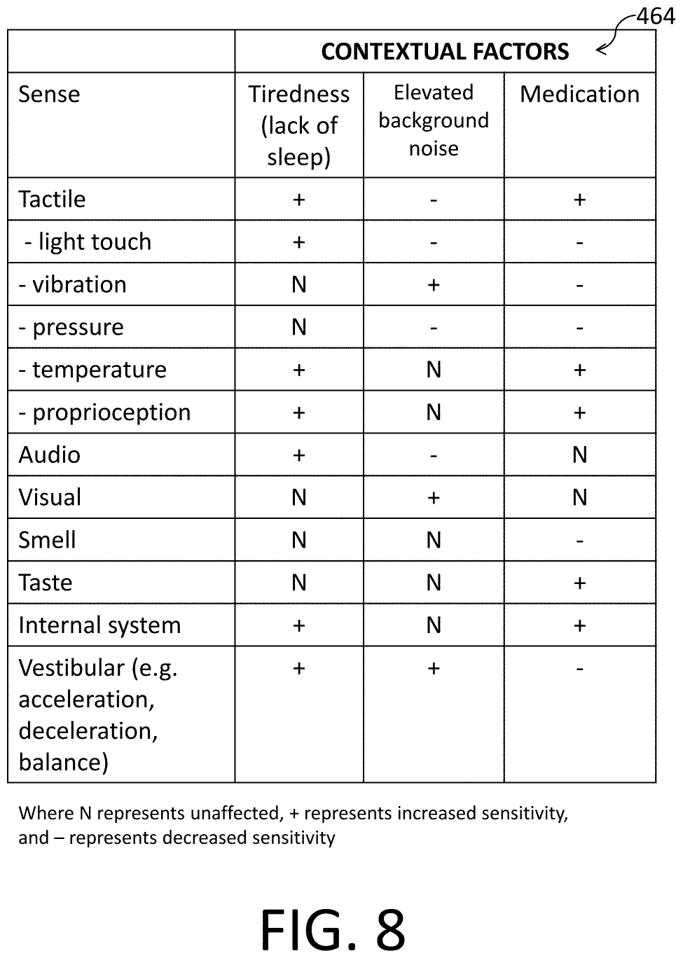

(iii) Contextual data 464 comprises measured and/or input values for the contextual factors. Contextual factors and contextual data 464 for the user 240 in one embodiment of the present technology are shown in FIG.8.

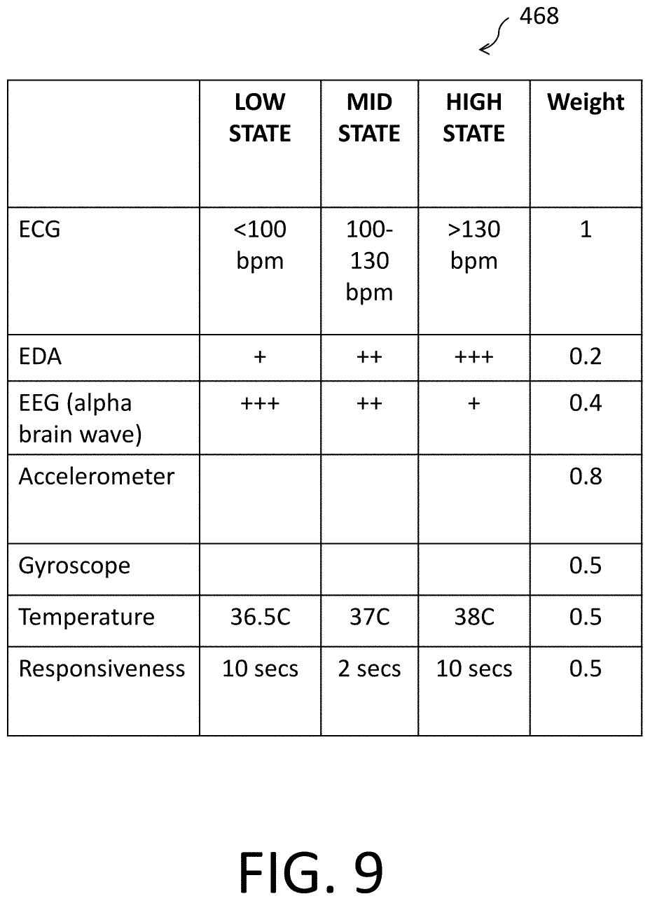

(iv) Physiological parameter profile 468. Physiological parameter profile of the user 240 can include information on the baseline physiological parameter ranges of the user 240 in each of the arousal states of the user 240 (defined for example as LOW state, MID state and HIGH state). FIG. 9 illustrates the physiological parameter profile 468 for the user 240 according to one embodiment of the present technology. This data 468 is used to determine a current arousal state for the user based on measured physiological data and can also be used to monitor the user 240.

(v) Physiological parameter weights 470

In certain embodiments, each of the physiological parameters from the physiological parameter profile 468 of (iv) above is assigned a weight (“physiological parameter weight”) based on the pertinence of that particular parameter as an indicator of an arousal state. The physiological parameter weights may be included in the physiological parameter profile. The weights can be predetermined. For example, if a particular parameter such as sweat levels or heart rate does not vary much from one arousal state to another then these parameters will be assigned a lower weight. Another example is if the user 240 has very high sweat levels, that physiological parameter of sweat will be assigned a lower weight than another physiological parameter which is more variable with arousal state. In another example, the assigned weight may also be an indicator of the relative reliability of the physiological parameter as an indicator of an arousal state. For example, if the user 240 has levels of movement ranges that are inconsistent or not representative of their arousal state, then the movement parameter will be assigned a lower weight. In the example of FIG. 9, weights, on a scale of 0 to 1, have been assigned to each of the physiological parameters. It will be appreciated that any scale can be used for the weights.

(vi) Contextual factor weights

In certain embodiments, at least some of the contextual factors may include a relative weight. For example, if the user is tired, regardless of any other factor this may place them by default into the LOW arousal state therefore this contextual factor is assigned a high weight. In another example, the tiredness of the user may affect the sense of touch more than the visual sense. Therefore, tiredness as a contextual factor is assigned a higher weight than other contextual factors. The contextual factor weights can be predetermined. The contextual factor weights can be included in the contextual factor profile.

(vii) Disorder profile

Disorder profile comprises information on a disorder or condition of the user and a level of disorder of the user in that condition, if applicable. For example, for a user with autism spectrum disorder, the disorder levels are selected from level 1 “requiring support”, level 2 “requiring substantial support” and level 3 “requiring very substantial support”. This information about the user can be used to cross-reference with other databases regarding disorder levels and typical sensory profiles and/or arousal state physiological parameter range profiles. Disorder or condition can refer to any cognitive or neurological condition affecting sensory input processing, such as a coma state, a habit state or an addictive state. Other conditions include Alzheimer's, or Parkinson's. It is to be noted that the Disorder Profile of the user may change over time, and will require updating. In certain embodiments, the Disorder Profile is related to a number of factors such as cumulative effects of stressors, the user's coping mechanism, and the user's support network. The disorder profile may include information regarding the verbal level of the user 240 (e.g. verbal, non-verbal).

(viii) Predetermined threshold data of the user. This can include information on certain parameters related to the user 240 which are indicative of intervention being required (such as application of a calibrated communication signal). For example, if the user 240 is determined to be in a certain arousal state for a certain period of time, this may indicate that they are “stuck” and require modulation to another arousal state. Another example, is a measured physiological parameter reaching a certain value, at which point intervention is deemed necessary, such as but not limited to a high body temperature, or involuntary limb flailing.

(ix) Interaction profile. The interaction profile of the user 240 is information regarding how the user 240 responds directly to specific communication signals and how this relates to the user's arousal states. In certain embodiments, it defines value ranges of interaction parameters with at least one arousal state of the user 240. This profile can be built through use, as the user 240 is exposed to different communication signals. In certain embodiments, the interaction profile includes various interaction parameters relating to direct user 240 response to the communication signal, such as an intensity of a direct user response, a duration of the direct response, a time delay of the direct response, or a frequency of the direct user response. The direct user response can be one or more of: the touch response of the user 240, a sound generated by the user 240, a movement of the user 240, a facial expression of the user 240, a brain signal of the user 240, and a flight action of the user 240, an action of the user 240 taking off the wearable device, and the like. The movement of the user 240 can be voluntary or involuntary. In certain embodiments, the interaction profile also includes user interaction weights which are predetermined weight allocations of the direct user input parameters. The Interaction profile can also include indicators of fail-safe levels for ending the application of the calibrated communication signal. The responses of the user 240 to specific communication signals include interactions with another user 260.

In certain embodiments, the user profile includes custom markers relating to a given task to be undertaken by the user. Tasks may include getting dressed, playing chess, studying, swimming, or self-regulating tasks such as nail-biting, rocking etc. Baseline values of physiological parameters and contextual factors relating to the custom markers may be established. Baseline values of all biomarkers relating to arousal state change or as indicators of stress are established by the set-up module.

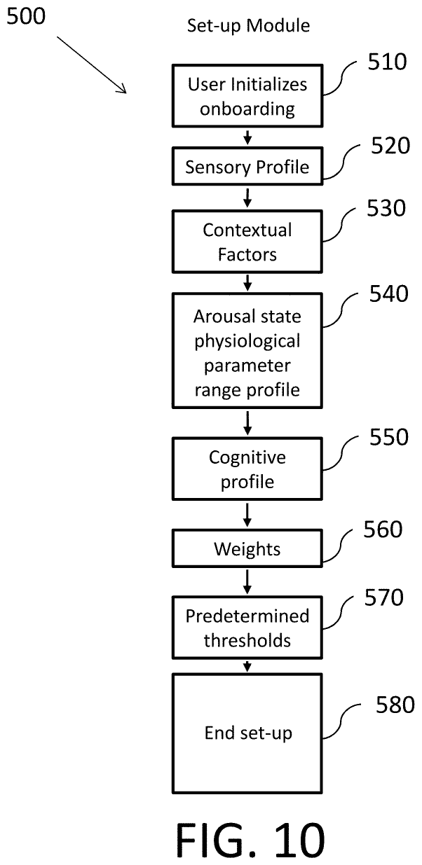

Turning now to FIG. 10, which illustrates one embodiment of the method executed by the set-up module. The method 500 is for collecting data related to the user and/or the environment. The method 500 starts by a step 510 in which a new user is onboarded. This step may include the creation of an internal ID. A time stamp may be entered or automatically generated at this time. Generally, the method 500 comprises establishing or obtaining the user profiles, which may be any one or more of the sensory profile 462, the disorder profile, the physiological parameter profile, the physiological parameter weights, contextual factor profile, contextual factor weights, interaction profile. In step 520, the sensory profile 462 of the user 240 is established. The method 500 may also proceed, at a step 530, with collecting data relating to contextual factors. At step 540, the method 500 may proceed with collecting data regarding the physiological parameter profile. Then, at step 550, weights may be assigned to one or more of the physiological parameters of step 540 and the contextual factors of step 530 (e.g. the physiological parameter weights and the contextual factor weights). At step 560, the sensory profile 462 is obtained. At step 570, the predetermined thresholds are obtained. At step 580, the set-up may end. The end of the method at step 580 may trigger the start of a method for determining the current arousal state by the arousal state module 420, or a method for monitoring the current arousal state by the monitoring module 450. It will be appreciated that certain of the steps 520 to 570 may be omitted. It will be appreciated that the steps 520 to 570 can be performed in any order or be performed consecutively. It will also be appreciated that the method 500 may comprise other steps for obtaining or establishing other user profiles.

In some embodiments, the set-up module 410 collects the data through one or more of a manual input of data, automatic collection of data, or semi-automatic collection of data. The data may be collected from a database, or may comprise, at least in part, data collected from measurement devices, sensors or other devices associated with the user 240. Certain data may be provided to the set-up module 410 by the machine learning module 460. This may include updated weights in step 560 or updated interaction profile data. The set-up module 410 may be arranged to perform the steps of the method 500 at predefined intervals, or after certain triggers, in order to update the data as necessary.

### The Arousal State Module 420

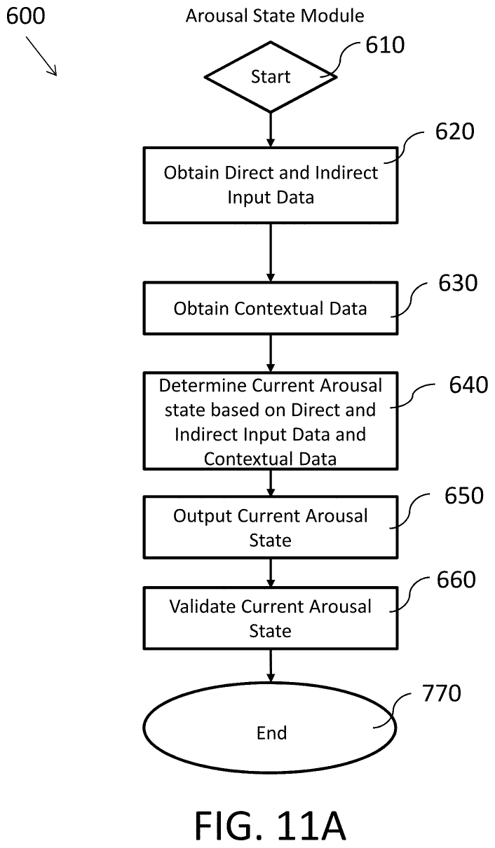

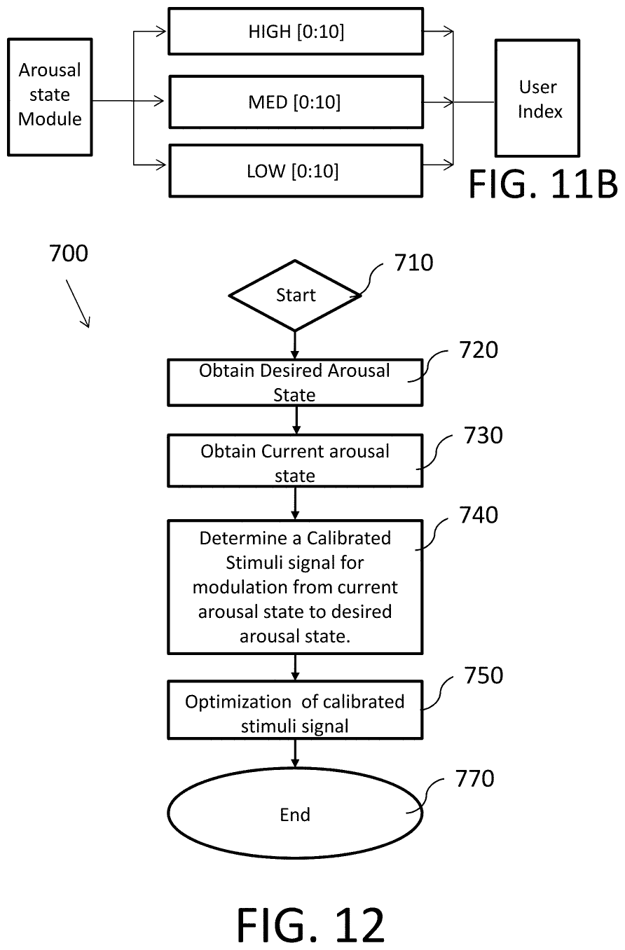

The arousal state module 420 will now be described with reference to FIGS. 11A and 11B. In the embodiment of FIGS. 11A and 11B, the arousal state module 420 is arranged to execute a method 600 for determining a current arousal state of the user 240. In this embodiment, the arousal states are the arousal state categories of HIGH, MID and LOW. In other embodiments, the arousal states include a further granulation within the current arousal state category. In this respect, each of the arousal states of HIGH, MID and LOW can be subdivided in any manner of useful ways, such as numerical scales (e.g. 1 to 10, 1 to 5, 0 to 3, 0 to 1 etc); letter scales (e.g. a to z, 1 to p); upper, middle and lower; and the like. In these embodiments, the current arousal state of the user thus determined will be in the form of L.M, where L defines the broader arousal state a category and M defines the position within the arousal state (e.g. the subdivision). This is termed herein as the “User Index”. In other embodiments, the current arousal state category or the user index will include a time coefficient which is indicative of an approximate time that the user has been in that arousal state. The current arousal state determination can thus be in the form of L.N or L.M.N, where L defines the broader arousal state a category, M defines the position within the arousal state category (e.g. the subdivision), and N defines the time within the arousal state category. The time coefficient can be a useful indicator of a transiency or a permanency in a particular arousal state and the type of modulation that may be required to move away from the current arousal state towards a future arousal state.

The method 600 starts at step 610. The start of method 600 may commence based on a number of different triggers such as instructions to commence being received by the controller (such as from the user or the other user), a predetermined time trigger, a predetermined time interval trigger, an input data trigger such as when a value of the input data reaches or exceeds a threshold value, or a contextual data trigger such when a value of the contextual data reaches or exceeds a threshold value. The trigger to commence the method 600 may also be received on completion of the method 500 of the set-up module 410, or on a trigger from the monitoring module 450 which in certain embodiments monitors the input data and determines whether the method 600 should commence.

At Step 620, input data of the user 240 to a communication signal is obtained, the input data comprising direct input data and/or indirect input data, such as from the input devices 230 associated with the user 240. This input data may be pulled by the computer system 401, or pushed by the input devices 230 or the controller unit 210. In certain embodiments, the input data is responsive to a communication signal. The communication signal may be selected based on various factors such as those determined to induce a reaction in the user 240 which is helpful in identifying their current arousal state. In these embodiments, the input data can also include a rate of response. The communication signal may be provided to the user 240 in any manner, such as through the communication device 220, the input device 230, the monitoring device 250, or from the other user 260.

Accordingly, in these embodiments, the Step 620 may additionally include providing a communication signal to the user 240 before obtaining the input data, such as by sending instructions to the communication device 220 to provide the communication signal to the user 240. The communication signal may be defined by a signal amplitude, a signal frequency, a signal wavelength, a signal duration, a signal pattern, and a signal code. In certain embodiments, the communication signal is a frequency-based signal such as a sound, haptic, visual signal. The communication signal may be a plurality of different signals, such as a bi-haptic signal.

Optionally, the method 600 may include determining the communication signal to apply to the user 240 based on the user profile, and one or more of: the initial arousal state of the user, the physiological data, the contextual data, and a preference of the user. Therefore, in certain embodiments, the communication signal is likely to be different at different time points. This means that the communication signal is determined every time in certain embodiments. Alternatively, the communication signal may have been predetermined.

The determining the communication signal to provide to the user 240 may comprise determining one or more of: the signal type, the signal amplitude, the signal frequency, the signal wavelength, the signal duration, the signal code and the signal pattern. In certain embodiments, the determining the communication signal comprises executing a trained machine learning algorithm, the machine learning algorithm having been trained based on at least one of the following training inputs: the initial arousal state of the user, the desired arousal state of the user, the sensory profile of the user, the contextual factor profile of the user, the contextual factor weights, the physiological parameter profile, the physiological parameter weights, the disorder profile of the user, and the interaction profile of the user.

The obtaining the indirect input data could comprise a continuous monitoring of physiological parameters of the user 240, through the wearable device 230 for example. The input data may also include an initial arousal state of the user, obtained for example through a self-evaluation of the user 240, or from the other user 260 or another third party. The method may include subsequent processing of the obtained input data, such as by natural language processing.

The obtaining the input data relating to the direct response of the user 240 comprises, in certain embodiments, obtaining user input values of the direct response of the user, the user input values relating to one or more user input parameters of: an intensity of the direct user response, a duration of the direct user response, a time delay of the direct user response, a location of the direct user response, a frequency of the direct user response, and a pattern of when the responses are given over a known time period (e.g. during stressful tasks). The direct response of the user 240 can comprise one or more of a touch response, a sound response, a kinetic response, a brain signal response, a pupil size or change in pupil size, a breath response, and a facial response. The physiological response of the user 240 can comprise one or more of a heart rate, a breathing rate, a blood flow, a sweat analysis, a measure of movement, an electrical brain signal, a temperature, a breath analysis, and one or more biomarkers of stress.

The input data of the user 240 can be obtained from at least one of: the communication device 220, such as the wearable haptic device associated with the user and operably connected to the module, the input device 230 associated with the user or with the other user 260, or directly from the other user 260.

At optional step 630, contextual data 464 is obtained. The contextual data 464 may be obtained from the input devices 230, for example, in real-time. The contextual data 464 may comprise environmental data measured by the input devices 230 (e.g. temperature, humidity etc) or information about the user input into the computer system 401 (e.g. tiredness, medication, dosage etc). The obtaining the direct and indirect user input and the contextual data may be obtained as a single step.

In step 640, the current arousal state is determined based on a function or correlation of the input data (i.e. direct input data, and/or indirect input data), the contextual data, and the user profile. The weights of the various parameters in the input data (e.g. physiological data profile) and the contextual data may be taken into account. In certain embodiments, the determination comprises a comparison of the input data with the physiological parameter profile. In certain embodiments, the determination comprises applying the physiological parameter weights. In certain embodiments, the determination comprises comparing the contextual data with the contextual factor weights. The determination can also comprise taking into account the interaction profile of the user 240.

The determination can comprise applying a trained algorithm at Step 640. The trained algorithm can be trained and/or implemented by the machine learning module 460. The algorithm may be user specific or may be categorized by other factors, such as disorder profile, or an age of the user, etc.

For example, referring back to the Example of FIG. 9, if an ECG measurement showing the user 240 heart rate of over 130 bpm together with a skin EDA measurement of the user of 4.2 μS (microsiemens) is obtained, it can be determined that the current arousal state of the user is the HIGH arousal state. However, as the EDA parameter has a lower weight associated with it for that particular user, taking into account the lower weight of the EDA parameter, may determine that the current user arousal state is in fact in the MID arousal state. Contextual data may result in a determination that if the background noise is above 60-80 dBA, the user 240 will almost always be in the HIGH arousal state based on the user's contextual factor profile. This factor may then result in a determination of HIGH state as the current arousal state.

An example algorithm to be used in step 640 to determine the Current Arousal State

or the User Index, is in the form:

Current Arousal State(e.g. L.M.N or L.N=function(aX, bY, cZ) Eqn.1

where X, Y and Z are input data and/or contextual data, and a, b and c are the respective weights for each of the parameters.

In certain embodiments, the method 600 includes optional Step 650 comprising outputting the determined current arousal state, such as a current arousal state output, to the monitoring device 250 of the other user 260 or one of the communication devices associated with the user 240. The current arousal state output can be provided in any form, such as writing on a screen, a pictorial representation, a verbal output. The form can be selected based on the sensory profile 462 of the user 240, or by the user as a user preference. In certain embodiments, the determined current arousal state is provided to the user 240 only in the instance that the user 240 may be required to exercise self-regulation to maintain an arousal state. For example, if the method 600 detects that the current arousal state is close to or moving towards an undesired arousal state (e.g. a crisis), the method 600 comprises providing an alert to the user 240. In certain embodiments, the alert comprises an reproduction of the user's heart rate as a haptic signal applied through the wearable device.

In certain embodiments, the method 600 includes optional Step 660 comprising validating the determined current arousal state. The validating can be performed in a number of different ways. In certain embodiments, the validation step 660 comprises direct validation by the user or the other user of the determined current arousal state in the form of direct validation input. The direct validation input may be responsive to a display of the determined current arousal state such as on any one or more of the communication devices, the input devices 230 or the monitoring devices 250. The validation input from the other user 260 or the user 240 can be provided through the monitoring device 250 or the input device 230. The validation input from the user 240 can be direct through an input device (e.g. touching a touch screen, pointing to a screen, talking, etc). The validation input may be a combination of direct input from the user 240 and direct input from the other user 260. For example, the user 240 may provide verbal input which the other user 260 enters digitally into the computer system 401. Alternatively, the computer system 401 may include speech-to-text capabilities which can convert audio data to text data. The validation input may be accessed by the machine learning module 460 for continued development of the current arousal state determination algorithm.

In certain embodiments, the validation step 660 comprises indirect validation through the application of a validation communication signal and a measurement of an indirect validation response. The validation communication signal can include any sensory input such as, but not limited to, a haptic signal, an audio signal, a visual signal, a telekinetic signal, a movement signal, an olfactory signal, an electrical signal, a magnetic signal, a piezometric signal, and combinations of the same. The indirect validation response can include a measured physiological response, such as those defined in the indirect user input. The validation step 660 may comprise execution of a validation method comprising applying a validation communication signal, obtaining a validation response, determining from the validation response whether the validation response is indicative of an expected response of the user when the user is in a particular arousal state. The expected responses for various communication and for various arousal states may be obtained during the execution of the method 500 by the set-up module 410, or may be included in a database such as the user profile database 294. The method 600 ends at Step 670.

### The Calibration Module 430

The calibration module 430 will now be described with reference to FIG. 12. In the embodiment of FIG. 12, the calibration module is arranged to execute a method 700 for determining a calibrated communication signal for the user 240. The calibrated communication module could be for modulating a current arousal state of the user 240 to a desired arousal state of the user 240, for maintaining the current arousal state of the user 240, for improving performance of a given task, or any other purpose. The method 700 starts at step 710. The method 700 may commence based on a number of different triggers such as instructions to commence being received by the controller (such as from the user or the other user), a predetermined time trigger, a predetermined time interval trigger, a predetermined arousal state trigger based on the current arousal state of the user, an input data trigger such as when a value of the input data reaches or exceeds a threshold value, or a contextual data trigger such when a value of the contextual data reaches or exceeds a threshold value. The trigger to commence the method 600 may also be received from the monitoring module which in certain embodiments monitors the input data, the contextual data, and the current arousal state of the user 240 and determines whether the method 600 should commence.

In Step 720, the method 700 comprises obtaining the current arousal state of the user. This may be obtained from the arousal state module 420, or be obtained in any other way such as from the other user 260 through the monitoring device 250.

In Step 730, the method 700 comprises obtaining the desired arousal state of the user. The desired arousal state may be the same or different as the current arousal state. For example, in certain embodiments, the method 700 may be used to maintain a user in the current arousal state and so the desired arousal state and the current arousal state may be the same. In other examples the method 700 may be used to modulate the user from a HIGH arousal state to a MID arousal state in order to commence a therapy session. In other examples, the method 700 may be used to modulate the user from a LOW arousal state to a HIGH arousal state, to enable the user 240 to engage in sporting activities. In certain embodiments, the current user arousal state and/or the desired arousal state inputs may include the granularity described above and be in the form of L.M.N or L.N. In some embodiments, modulation within a category of arousal state may be desired. In certain embodiments, the desired arousal state comprises a self-regulated state of the user 240.

In Step 740, the method 700 comprises determining the calibrated communication signal. The calibrated communication may comprise one or more of the following signal parameters: a type of communication signal, an intensity of communication signal, a frequency of communication signal, a wavelength of communication signal, a duration of communication signal, a pattern of communication signal, a sequence of communication signal, a rate of change of the communication signal, etc. The determining the calibrated communication signal comprises determining at least two calibrated communication signals, the at least two calibrated communication signals differing from one another in terms of one or more of a type of signal, an amplitude of signal, a frequency of signal, a duration of signal, an harmonic in the signal, a resonance in the vibration, a rate of change of signal, and a signal pattern, a signal sequence of signal and rate of change of the sequence of vibration. The communication signals are haptic signals, in certain embodiments.

In certain embodiments, the calibrated communication comprises one or more of an action, a spoken word, an auditive prompt, a visual prompt, a choreographic gesture, a musical tone delivered by a virtual character to be depicted on a screen of an electronic device.

The determination of the calibrated communication signal is a function of at least one of the user profile data (e.g. sensory profile, contextual factor profile, disorder profile), the current arousal state, contextual data, and user preference. The determination of the calibrated communication signal can comprise applying a trained algorithm at Step 740. The trained algorithm can be trained and/or implemented by the machine learning module. The algorithm may be user specific or may be categorized by other factors, such as disorder profile. The calibration module may also access data relating to measured responses of the user to various communication signals, which may be stored in the user profile database, the training model database, or another database. In certain embodiments, user preferences of the calibrated communication signal are obtained. For example, if the user prefers communication through a virtual character displayed on a screen of a device, the method may include obtaining the user preference relating to the virtual character, such as one or more of the personae, the display colour, language, the speech pattern, speech speed, and speech volume of the virtual character.

In certain embodiments, the determining the calibrated communication signal comprises receiving a desired communication to be communicated to the user 240 from the other user 260, transforming the desired communication using parameters determined by the calibrated communication signal. The method may then continue with sending instructions to the input device 230 or the communication device 220 to provide the desired communication in the transformed manner and in accordance with the determined calibrated communication signal. The desired communication can be a question, a statement, a phrase, a command, a custom marker, etc. Transforming the desired communication can mean slowing down the speech, applying a haptic signal at the same time as the virtual character delivering the desired communication, and the like.

The method 700 further comprises, in certain embodiments, monitoring the arousal state of the other user 260, and further transforming the desired communication if it is detected that the arousal state of the other user 260 is not within a predetermined arousal state. This is referred to herein as “co-regulation”. For example, if it is detected that the other user 260 is nervous or tired on one particular occasion when interacting with the user 240, as the user 240 may sense this, the calibrated communication signal or the communication signal may be adapted appropriately.

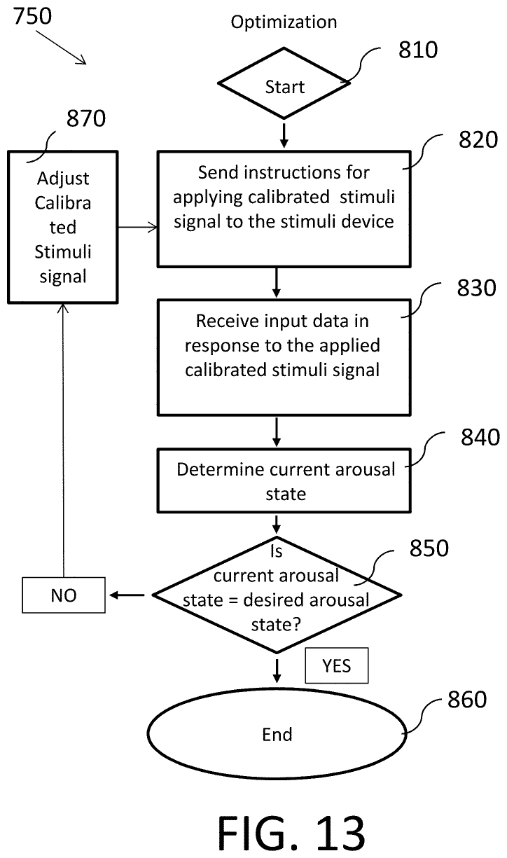

In optional step 750, an optimization of the calibrated communication signal is performed. In certain embodiments, optimization is performed using a looped pipeline process. Certain embodiments of the method executed in the optimization step 750 are shown in FIG. 13. The optimization method of step 750 starts at step 810. At step 820, the optimization method comprises sending instructions for applying the calibrated communication signal to the communication device 220. This includes determining, from the calibrated communication signal, the appropriate communication device 220, and sending instructions to the appropriate communication device 220. For example, the calibrated communication signal determined at Step 740 may comprise: a haptic signal with a certain signal signature of wavelength, frequency, amplitude, duration etc., together with an audio signal with a certain signal signature of audio frequency, loudness, duration etc. Therefore, at Step 820, the instructions for applying the calibrated haptic communication signal is sent to the communication device 220 which is the haptic device, and instructions for applying the calibrated audio communication signal is sent to the communication device 220. It will be appreciated that the calibrated communication signal can be any type of signal, and the instructions can be provided to the appropriate communication device appropriate for applying the calibrated communication signal. In certain embodiments, the calibrated communication signal is generated without input from the user or the other user, or anyone else, i.e. the calibration is automatic.

At step 830, input data, responsive to the applied calibrated communication signal, is received. The current arousal state is determined using the input data, according to the method 600 as described earlier with reference to FIGS. 11A and 11B. If the current arousal state is the same as the desired arousal state the optimization method 750 ends at step 860 and the calibration method (of FIG.12) ends at step 770. If the current arousal state is determined to not be the same as the desired arousal state, the optimization method 750 continues at step 870. In step 870, the calibrated communication signal is adjusted. The adjustment required can be implemented by a machine trained algorithm via the machine-learning module 460. These steps are iterated until the current arousal state is determined to be the same as the desired arousal state. Further inputs into the optimization include any data relating to the trigger of the fail-safe level and the applied calibrated communication signal. Other inputs include input data from the other user 260 regarding the arousal state of the user 240.

The determined communication signal and the adjusted communication signal can be stored in a database, such as the user profile database 294 or the training model database 296.

### Modulation Module 440

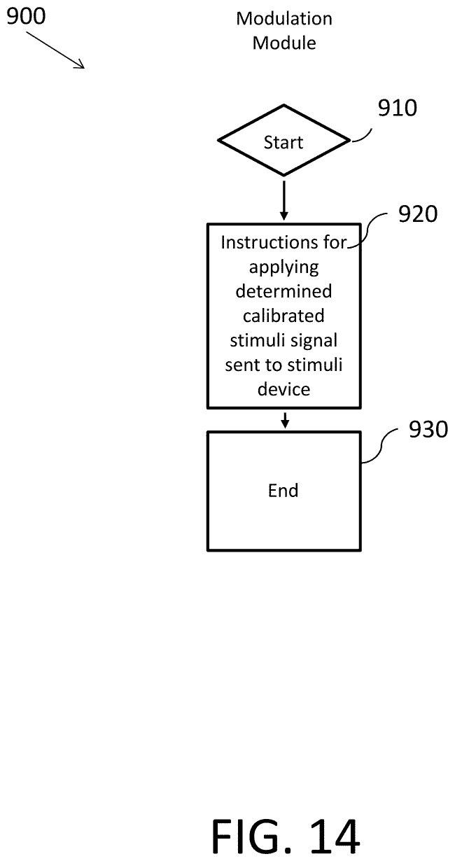

The modulation module 440 is arranged to execute a method 900 for modulating an arousal state of the user (shown in FIG.14). The method starts at step 910. At step 920, instructions for applying the determined calibrated communication signal is sent to the appropriate communication device 220. The method 900 ends at step 930.

In certain embodiments, the determined calibrated communication signal is retrieved from the database, such as the user profile database 294 or the training model database 296. In certain other embodiments, the determined calibrated communication signal is retrieved from the calibration module 430.

In certain embodiments, the method 900 includes a verification step by the other user 260 before the instructions to apply the determined calibrated communication signal is sent to the communication device 220.

In certain embodiments, the determined calibrated communication signal is an action and/or a verbal signal from a virtual character, such as an avatar. The virtual character may be an image presented on a screen of the input device 230, the communication device 220 or the monitoring device 250, for example. The virtual character may also be a virtual image or a three-dimensional figure. For example, the determined calibrated communication signal may be a lexicon from a tool box of communication signals, such as the virtual character saying a specific phrase in a soothing voice whilst rocking from side to side, or the virtual character holding out his hand whilst saying “stop” loudly. The tool box of communication signals may be grouped into certain groupings. Responses of the user 240 to the calibrated communication signal may be obtained through the same or different device presenting the virtual character to the user 240. The method 900 may continue with re-determining the calibrated communication signal according to the user response. It can be seen that in certain embodiments, a communication between the virtual character and the user 240 can be obtained with responsive calibrated communication signals from the avatar.

### Monitoring Module 450

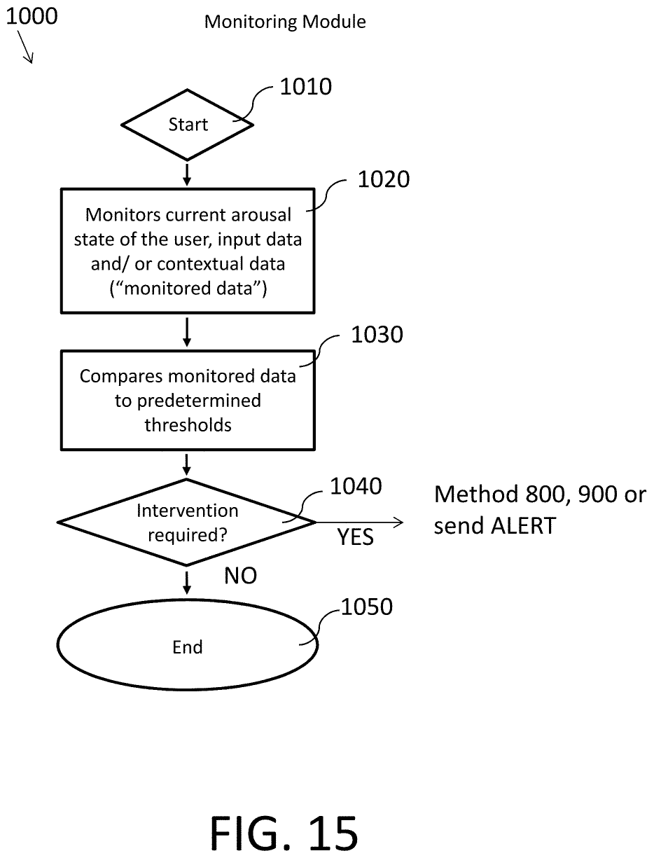

The monitoring module 450 will now be described with reference to FIG. 15. In the embodiment of FIG. 15, the monitoring module 450 is arranged to execute a method 1000 for determining whether the current arousal state of the user requires modulation.

The method 1000 starts at step 1010. At Step 1020, the monitoring module 450 monitors certain data (“monitored data”) such as the current arousal state of the user 240, input data (e.g. physiological data) or contextual data. The current arousal state data may have been determined by the arousal state module 420 or in any other way. The input data may include direct input data (e.g. direct user response) or indirect input data (e.g. measured physiological data). In certain embodiments, the monitoring step 1020 is conducted in real-time. In certain embodiments, the monitoring step 1020 is not in real-time.

At step 1030, the method 1000 determines whether an intervention to the user 240 is required. In certain embodiments, in step 1030, the monitored data is compared to predetermined thresholds. If the method 1000 determines that a predetermined threshold has been reached, a decision whether to intervene or not is reached in step 1040. If the decision reached is NO (no intervention required), the method 1000 ends at step 1050. If the decision reached is YES, the method 1000 may continue with the execution of method 700 to determine the current arousal state, method 900 to modulate the current arousal state, or by sending an alert.

In certain embodiments, determining whether modulation is required comprises monitoring the physiological data for the biomarkers or predictors of stress or arousal state changes (e.g. dysregulation events, crises).

The alert may be sent to the user 240 via one or more of the communication devices 220, or to the other user 260 via the monitoring devices 250 for example. The alert may consist of a desktop notification, a mobile notification, email, SMS and/or phone call. The predetermined thresholds could be with regard to any parameter and be user-specific, as determined during the set-up phase or otherwise.

### The Machine Learning Module 460

In some embodiments, the machine-learning module 460 may implement one or more machine-learning algorithms so as to leverage acquired data with data available in either the user profile database 294 and/or the training model database 296. In some embodiments, the machine learning module 460 can train and/or implement the algorithm for determining one or more of (i) the current arousal state, (ii) the calibrated communication signal, or the (iii) adjusted calibrated communication signal.

Examples of machine-learning algorithms implemented by the machine-learning module 460 may comprise, without being limitative, linear regression, logistic regression, decision tree, support vector machine, naïve bayes, K-nearest neighbors, K-means, random forest, dimensionality reduction, neural network, gradient boosting and/or adaboost. In some embodiments, the user profile database 292 and/or the training model database 296 may be implemented through database services such as, without being limitative, MySQL, PostGreSQL, MongoDB, MariaDB, Microsoft SQL Server, Oracle, Sybase, SAP HANA, MemSQL and/or IBM DB2.

In some embodiments, the algorithm for determining the current arousal state, the calibrated communication signal, and/or the adjusted calibrated communication signal may be modified by the computer system 401 based on the data collected by the set-up module 410, the calibration module 430 and/or the monitoring module 450.

In some embodiments, the machine-learning module 450 may aggregate data from multiple users to improve a relevancy and/or efficiency of the determination of the current arousal state and the calibrated communication signal for arousal state modulation.

In certain embodiments, the machine learning module 450 may enable co-regulation of the user 240 and the other user 260. Co-regulation in certain embodiments comprises an adjustment of the communication signal or the calibrated communication signal based on the arousal state of the other user 260 (when present).

By means of certain embodiments of the methods executed by the calibration module 430 and the modulation module 440, an optimized modulation of arousal state can be achieved. In certain embodiments, optimized modulation can mean the maintenance of a desired arousal state. In certain embodiments, optimized modulation can mean changing arousal states as quickly as possible. In certain embodiments, optimized modulation can mean changing arousal states or maintaining arousal states with an efficient extent of intervention in the form of communication signals.

In certain embodiments, the machine-learning module 450 is arranged to train an algorithm for determining the arousal state of the user based on at least one training input comprising input data, physiological parameter profile of the user, interaction profile of the user, the physiological parameter profile defining value ranges of the one or more physiological parameters within at least one arousal state of the user, and the interaction profile defining value ranges of interaction parameters with at least one arousal state of the user.

In one embodiment, the machine-learning module 450 is arranged to train an algorithm for determining the calibration communication signal based on various training inputs including one or more of a current arousal state of the user, a desired arousal state of the user, a disorder profile of the user, user data responsive to a communication signal, and user data responsive to a calibrated communication signal.

sApplications of Methods and Systems of the Present Technology

As described above, certain embodiments of the methods 500, 600, 700, 750, 900, 1000, and systems 400 of the present technology are used as a regulation tool for users' with conditions affecting arousal state regulation conditions, such as autism, ADHD, Alzheimer's, Parkinson's, Acute Traumatic Brain Injury, and the like. Certain embodiments can be considered as a tool to enhance or improve the homeostatic mechanism of the user 240. Other uses are listed below, which is a non-exhaustive list:

### Treatment Testing

Other applications of the methods and systems described herein are for the monitoring of treatments or therapies including drugs/medication. Clinical trials are one such setting where the arousal state of the user is monitored in parallel with the drug/treatment being tested. Therefore, in certain non-limiting embodiments, the method further comprises monitoring of the arousal state of the user, in any manner as described herein: before, during or after a treatment regime. The treatment regime may comprise the administering of a drug or a molecule for testing efficacy in treatment or control of symptoms of any condition, such as autism, ADHD, and major depressive disorder. The drug or molecule may comprise an anti-depressant, anti-anxiety, or an ADHD stimulant, for example. The monitoring of the arousal state of the user may be a continuous monitoring during any of the above-defined treatment periods. Continuous monitoring before commencement of the treatment can establish a baseline for the user. Continuous monitoring tracks the “reactivity” of the user to the treatment. In certain embodiments, the comparison of the user arousal state, and the treatment provided, can provide a more accurate picture of the treatment efficacy and potential side effects. Evaluating physiological responses to the treatment (such as blood analysis, plasma analysis, urine analysis etc) provide an even more complete picture. For example, in certain embodiments, the method comprises determining an appropriate dosage of a drug/molecule for a given treatment, by assessing half life of the drug/molecule in the user, together with the arousal state monitoring.

Advantageously, in certain embodiments, the arousal state monitoring provided by embodiments of the present technology provides a more accurate determination of the actual arousal state of the user compared to a self-evaluation or compared to a third party observation of the user. For psychoactive medications particularly, such as anti depressants, anti anxiety medications, ADHD stimulants, evaluation of efficacy relies on self reporting by the user or observation by third parties (e.g. the other user 260).

For example, children with ASD may sometimes appear to be in a low arousal state, when in fact they are actually in a HIGH arousal state and overwhelmed, the lower apparent activity being a shut-down mechanism to cope with overstimulation.

Additionally, users with Autistic Spectrum Disorder tend to have poor interoception of their stress levels, so their perception and self report could be unreliable at times. The obtaining and use of the user's physiological input in determining the arousal state in certain embodiments, provides a more accurate determination of the treatment efficacy compared to prior art methods.

### Treatment Compliance

Certain embodiments of the present methods and systems are used to improve or increase compliance of the user to a particular treatment, such as taking medication. The communication device 220, such as the wearable device, provides alerts to the user 240 for treatment adherence, such as taking a medication.

### Addiction Management

Certain embodiments of the present methods and systems are used as an addiction management tool for the user 240. In these embodiments, the arousal state of the user can be monitored, and a change in the arousal state can indicate that the user is experiencing a craving to fulfill the addiction. When this occurs, the user can be informed to allow them to be self-aware and self regulate. The detection of the craving can also initiate an addiction treatment, which could be provided by the wearable device, such as cognitive therapy, reinforcement therapy, etc. Additional user interfaces can be provided to record patient's self assessment.

### Arousal State Self-Regulation

Certain embodiments of the present methods and systems are used to enable a user to self-regulate an arousal state. In these embodiments, the determined arousal state is communicated to the user in any suitable way, such as through the communication device 220 in the form of any communication signal, such as an appropriate calibrated communication signal, through the virtual character depicted on the communication device 220, as a haptic signal provided to the user through the wearable device, or by the other user 260.

### Coma Monitoring

Certain embodiments of the present methods and systems are used to monitor an arousal state of a patient in a coma.

### Other

In other embodiments, the methods and systems described herein can be applied to users 240 in isolated situations such as space missions, military training, as well as to users 240 in simulations of these situations using virtual reality and augmented reality exercises and the like. In other embodiments, the methods and systems described herein can be applied to home caregiving situations such as to the elderly or infirmed.

Identification of equivalent methods and systems are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

### Example 1

### Set-Up for Calibrated Communication Module

A set-up of the system was performed prior to the use of the system for providing a calibrated communication signal to a number of users 240, all children with Autistic Spectrum Disorder. Each user 240 was asked to select a number of preferences regarding the calibrated communication signal, in this case a virtual character displayed on the screen of a communication device 220 in the form of a tablet device. Each user 240 selected one of a possible number of options relating to the personae of the virtual character, the speed of speech of the virtual character, and the colour of the virtual character. Baseline data was collected for each user 240 using established and adapted occupational therapy tools, including ABAS, CBCL, ABC, SFA, GAS, EDI. Each user had a predetermined task selected for evaluation (see Example 2). Therefore, baseline data was also collected regarding the duration of performing the given task for each user. The tasks included writing text, doing maths, dressing and transition between two tasks.

### Example 2

### Applying Calibrated Communication Module During a Given Task

For each user and their given task, the system determined the calibrated communication signal to provide to the user during performance of the task for the purposes of: maintain the user's arousal state during the given task, to reduce the user's risk of flight during the given task and to increase engagement of the user 240 during the given task. The calibrated communication signal was provided to each of the user's 240 during their performance of the given task using the virtual character. The timing of the intervention using the calibrated communication signal was determined according to the continuous monitoring of the input data. Indicators and levels of stress were determined in the input data (e.g. heart rate increase, flapping increase) and when the input data indicated these levels for the identified indicators, the calibrated communication signal was applied.

The results showed that for all the users 240, providing the user 240 with the calibrated communication signal during their performance of the task, reduced their instances of flight, increased their engagement, resulted in less requests for help from the user.

While the above-described implementations have been described and shown with reference to particular steps performed in a particular order, it will be understood that these steps may be combined, sub-divided, or re-ordered without departing from the teachings of the present technology. At least some of the steps may be executed in parallel or in series. Accordingly, the order and grouping of the steps is not a limitation of the present technology.

It should be expressly understood that not all technical effects mentioned herein need to be enjoyed in each and every embodiment of the present technology.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1-54. (canceled)

55. A method for determining an arousal state of a user, the method executed by a processor of a computer system, the method comprising:
obtaining a user profile of the user,
obtaining multimodal input data relating to the user, wherein the multimodal input data comprises:
physiological data of the user, wherein the physiological data is obtained by a wearable device worn by the user,
a direct response of the user to a communication signal, wherein the direct response comprises one or more user input parameters, the user input parameters comprising one or more of an intensity of the direct response, a duration of the direct response, a time delay of the direct response, a location of the direct response, a frequency of the direct response, a pattern of when direct responses are given over a known time period, and a selection of an indicator from a plurality of indicators, and
a timestamp corresponding to one or both of the physiological data and the direct response of the user; and
determining the arousal state of the user based on a relationship between:
the multimodal input data, and
the user profile of the user.

56. The method of claim 55, wherein the determining of the arousal state comprises determining, by a trained machine learning algorithm, the arousal state of the user, the machine learning algorithm trained to determine the arousal state of the user based on at least one of:
the multimodal input data,
the user profile,
an initial arousal state of the user, and
contextual data regarding contextual factors relating to different available arousal states of the user,
wherein the user profile comprises data relating to one or more of: a physiological parameter profile of the user, a disorder profile of the user, a sensory profile of the user, a contextual factor profile of the user, or an interaction profile of the user.

57. The method of claim 56, wherein the user profile comprises contextual factor weights for the user and physiological parameter weights for the user, and wherein the different arousal states comprise the arousal state of the user.

58. The method of claim 55, wherein the physiological data of the user comprises one or more of: a heart rate, a breathing rate, a blood flow, a sweat analysis, a measure of movement, an electrical brain signal, a temperature, a breath analysis, electrodermal activity, and one or more biomarkers of stress or cognitive impairment.

59. The method of claim 55, wherein the direct response of the user comprises a touch response, a sound response, or a kinetic response and corresponding contextual data.

60. The method of claim 55, wherein the arousal state comprises one of a plurality of available arousal state categories, the method comprising determining a user index, the user index being indicative of a given arousal state category and a relative position within the arousal state category.

61. The method of claim 55, further comprising determining the communication signal to provide to the user, the determining the communication signal being based on the user profile, an initial arousal state of the user, contextual data, and a preference of the user.

62. The method of claim 55, further comprising determining a calibrated communication signal effective to modulate the user from the arousal state to a desired arousal state, the determining the calibrated communication signal being based on the user profile of the user, wherein the calibrated communication signal is a haptic signal, a light signal, a sound signal, an olfactory signal, a visual kinetic signal, a sensory kinetic signal, a magnetic signal, an electric brain signal, or a piezometric signal, and wherein determining the calibrated communication signal comprises executing, by the processor, a trained machine learning algorithm, the machine learning algorithm having been trained on at least one of the following training inputs: the arousal state of the user, the desired arousal state of the user, a disorder profile of the user, an interaction data profile of the user, a sensory profile of the user, a contextual factor profile of the user, contextual factor weights corresponding to the user, a physiological parameter profile of the user, physiological parameter weights corresponding to the user, and the multimodal input data.

63. The method of claim 55, wherein the direct response is obtained by the wearable device.

64. The method of claim 55, wherein the communication signal or the direct response is input by a third party.

65. A method for determining a change in an arousal state of a user, the method executed by a processor of a computer system, the method comprising:
retrieving, from a memory of the computer system, a user profile of the user, wherein the user profile comprises:
a physiological parameter profile of the user, wherein the physiological parameter profile comprises a plurality of physiological parameters, and wherein the physiological parameter profile comprises, for each physiological parameter of the plurality of physiological parameters, an associated physiological value range, and
an interaction profile of the user, wherein the interaction profile indicates how the user has responded previously to specific communication signals, and wherein the interaction profile of the user comprises one or more of: user input values of a response to the specific communication signals, an intensity of the response to the specific communication signals, a duration of the response to the specific communication signals, a time delay of the response to the specific communication signals, a location of the response to the specific communication signals, a frequency of the response to the specific communication signals, or a pattern of when responses are given to the specific communication signals;
receiving data comprising:
an indication of a response to a communication signal, and
physiological data of the user, wherein the physiological data is measured by a wearable device worn by the user; and
determining the change in the arousal state of the user based on comparing the data measured by the wearable device to the user profile; and
outputting an indication of the arousal state of the user.

66. The method of claim 65, wherein determining the change in the arousal state of the user comprises:
determining a value of a physiological parameter in the data measured by the wearable device; and
determining that the value is outside of a physiological value range corresponding to the physiological parameter in the user profile of the user.

67. The method of claim 65, wherein the wearable device comprises one or more sensors for measuring the response to the communication signal, and wherein the indication of the response to the communication signal comprises an indication of an intensity of the response to the communication signal, a duration of the response to the communication signal, a time delay of the response to the communication signal, or a frequency of the response to the communication signal.

68. The method of claim 65, wherein the indication of the response to the communication signal, comprises an indication of a response to a questionnaire completed by the user or a third-party.

69. The method of claim 65, wherein the specific communication signals were output by the wearable device.

70. The method of claim 65, further comprising sending instructions to the wearable device to provide the communication signal to the user, wherein the communication signal is a frequency-based signal, and wherein the instructions comprise a signal amplitude, a signal frequency, a signal wavelength, a signal duration, a signal pattern, or a signal code.

71. The method of claim 65, further comprising determining, based on the arousal state of the user, a calibrated communication signal to output to the user, wherein the calibrated communication signal comprises a frequency-based signal defined by a signal type, a signal amplitude, a signal frequency, a signal wavelength, a signal duration, a signal code, or a signal pattern.

72. The method of claim 71, wherein determining the calibrated communication signal comprises determining, by a machine learning algorithm, the calibrated communication signal, and further comprising applying the calibrated communication signal until a desired physiological measure of the user is detected.

73. The method of claim 65, wherein the communication signal was output by the wearable device and wherein the response to the communication signal was input on the wearable device.

74. The method of claim 65, wherein the response to the communication signal was input by a third-party.
